(19) United States
(12) Patent Application Publication
(10) Pub. No.: US 2002/0048782 A1
(43) Pub. Date: Apr. 25, 2002

(54) PYK2 RELATED PRODUCTS AND METHODS

(76) Inventors: SIMA LEV, SAN CARLOS, CA (US); JOSEPH SCHLESSINGER, NEW YORK, NY (US)

Correspondence Address: FOLEY & LARDNER, WASHINGTON HARBOUR, 3000 K STREET N.W., SUITE 500, WASHINGTON, DC, 20007-5109, US

(21) Appl. No.: 08/987,689

(22) Filed: Dec. 9, 1997

Related U.S. Application Data
(60) Provisional application No. 60/032,824, filed on Dec. 11, 1996.

(51) Int. Cl. G01N 33/53; C12Q 1/68; C07H 21/04; C12P 21/06; C12N 5/00; C12N 5/02; C07K 1/00; C07K 14/00; C07K 17/00

(52) U.S. Cl. 435/69.1; 435/325; 530/350; 536/23.5; 435/7.1

(57) ABSTRACT

The present invention features a method for treatment of an organism having a disease or condition characterized by an abnormality in a signal transduction pathway, wherein the signal transduction pathway includes a PYK2 protein. The invention also features methods for diagnosing such diseases and for screening for agents that will be useful in treating such diseases. The invention also features purified and/or isolated nucleic acid encoding a PYK2 protein.

### RELATED APPLICATIONS

The present application is related to U.S. Ser. No. 60/032,824, filed Dec. 11, 1996, entitled to PYK2 RELATED PRODUCTS AND METHODS, by Lev et al. (Lyon & Lyon Docket No. 222/126). This application is also related to U.S. application Ser. No. 08/460,626, filed Jun. 2, 1995, which is a continuation-in-part application of U.S. patent application Ser. No. 08/357,642, filed Dec. 15, 1994, both of which are incorporated herein by reference in their entirety, including any drawings.

### INTRODUCTION

The present invention relates generally to a novel protein termed PYK2 and related products and methods.

### BACKGROUND OF THE INVENTION

None of the following discussion of the background of the invention is admitted to be prior art to the invention.

Cellular signal transduction is a fundamental mechanism whereby external stimuli that regulate diverse cellular processes are relayed to the interior of cells. One of the key biochemical mechanisms of signal transduction involves the reversible phosphorylation of tyrosine residues on proteins. The phosphorylation state of a protein is modified through the reciprocal actions of tyrosine phosphatases (TPs) and tyrosine kinases (TKs), including receptor tyrosine kinases and non-receptor tyrosine kinases.

Receptor tyrosine kinases (RTKs) belong to a family of transmembrane proteins and have been implicated in cellular signaling pathways. The predominant biological activity of some RTKs is the stimulation of cell growth and proliferation, while other RTKs are involved in arresting growth and promoting differentiation. In some instances, a single tyrosine kinase can inhibit, or stimulate, cell proliferation depending on the cellular environment in which it is expressed.

RTKs are composed of at least three domains: an extracellular ligand binding domain, a transmembrane domain and a cytoplasmic catalytic domain that can phosphorylate tyrosine residues. Ligand binding to membrane-bound receptors induces the formation of receptor dimers and allosteric changes that activate the intracellular kinase domains and result in the self-phosphorylation (autophosphorylation and/or transphosphorylation) of the receptor on tyrosine residues. Individual phosphotyrosine residues of the cytoplasmic domains of receptors may serve as specific binding sites that interact with a host of cytoplasmic signaling molecules, thereby activating various signal transduction pathways.

The intracellular, cytoplasmic, non-receptor protein tyrosine kinases do not contain a hydrophobic transmembrane domain or an extracellular domain and share non-catalytic domains in addition to sharing their catalytic kinase domains. Such non-catalytic domains include the SH2 domains and SH3 domains. The non-catalytic domains are thought to be important in the regulation of protein-protein interacions during signal transduction.

Focal adhesion kinase (FAK) is a cytoplasmic protein tyrosine kinase that is localized to focal adhesions. Schaller, et al., Proc. Natl. Acad. Sci. U.S.A., 89:5192-5196 (1992), incorporated herein by reference in its entirety, including any drawings; Cobb et al., Molecular and Cellular Biology, 14(1):147-155 (1994). In some cells the C-terminal domain of FAK is expressed autonomously as a 41 kDa protein called FRNK and the 140 C-terminal residues of FAK contain a focal adhesion targeting (FAT) domain. The cDNA's encoding FRNK are given in Schaller et al., Molecular and Cellular Biology, 13(2):785-791 (1993), incorporated herein by reference in its entirety, including any drawings. The FAT domain was identified and said to be required for localization of FAK to cellular focal adhesions in Hilderbrand et al., The Journal of Cell Biology, 123(4):993-1005 (1993).

A central feature of signal transduction is the reversible phosphorylation of certain proteins. Receptor phosphorylation stimulates a physical association of the activated receptor with target molecules, which either are or are not phosphorylated. Some of the target molecules such as phospholipase Cγ are in turn phosphorylated and activated. Such phosphorylation transmits a signal to the cytoplasm. Other target molecules are not phosphorylated, but assist in signal transmission by acting as adapter molecules for secondary signal transducer proteins. For example, receptor phosphorylation and the subsequent allosteric changes in the receptor recruit the Grb-2/SOS complex to the catalytic domain of the receptor where its proximity to the membrane allows it to activate ras. The secondary signal transducer molecules generated by activated receptors result in a signal cascade that regulates cell functions such as cell division or differentiation. Reviews describing intracellular signal transduction include Aaronson, Science, 254:1146-1153, 1991; Schlessinger, Trends Biochem. Sci., 13:443-447, 1988; and Ullrich and Schlessinger, Cell, 61:203-212, 1990.

Several protein tyrosine kinases are highly expressed in the central nervous system and there is evidence that protein phosphorylation plays a crucial regulatory role in the nervous system. Neurotrophic factors that control the differentiation and maintain the survival of different types of neuronal cells mediate their biological effects by binding to and activating cell surface receptors with intrinsic protein tyrosine kinase activity. Furthermore, protein phosphorylation is a key regulatory mechanism of membrane excitability and ion channel function.

Tyrosine phosphorylation regulates the function of several ion-channels in the central nervous system. Protein kinase C (PKC) can regulate the action of a variety of ion channels including voltage-gated potassium channels, voltage dependent sodium channels as well as the nicotinic acetycholine receptor. The action of the NMDA receptor can be modulated by protein-tyrosine kinases and phosphatases. Moreover, tyrosine phosphorylation of the nicotine acetylcholine receptors (AchR) increases its rate of desensitization, and may play role in regulation of AchR distribution on the cell membrane. Another example is the delayed rectifier-type K+ channel, termed Kv1.2 (also called RAK, RBK2, RCK5 and NGKI). This channel is highly expressed in the brain and cardiac atria, and can be regulated by tyrosine phosphorylation. Tyrosine phosphorylation of Kv1.2 is associated with suppression of Kv1.2− currents. Suppression of Kv1.2 currents was induced by a variety of stimuli including carbachol, bradykinin, PMA and calcium ionophore.

The Ras/MAP kinase signal transduction pathway is highly conserved in evolution and plays an important role in the control of cell growth and differentiation. The MAP kinase signalling pathway in PC12 cells can be activated by NGF, by peptide hormones that activate G-protein coupled receptors, by phorbol ester as well as by calcium influx following membrane depolarization. However, the mechanism underlying activation of the Ras/MAP kinase signaling pathway by G-protein coupled receptors as well as by calcium influx are not known.

Shc is involved in the coupling of both receptor and non-receptor tyrosine kinases to the Ras/MAPK signalling pathways. Overexpression of Shc leads to transformation of 3T3 cells and to neuronal differentiation of PC12 cells. Moreover, Shc induced differentiation of PC12 cells is blocked by a dominant mutant of Ras indicating that Shc acts upstream of Ras. Tyrosine phosphorylated Shc can activate the Ras signaling pathways by binding to the SH2 domain of the adaptor protein Grb2 that is complexed to the guanine nucleotide releasing factor Sos via its SH3 domains.

Signal transduction pathways that regulate ion channels (e.g., potassium channels and calcium channels) involve G proteins which function as intermediaries between receptors and effectors. Gilman, Ann. Rev. Biochem., 56:615-649 (1987); Brown and Birnbaumer, Ann. Rev. Physiol., 52:197-213 (1990). G-coupled protein receptors are receptors for neurotransmitters, ligands that are responsible for signal production in nerve cells as well as for regulation of proliferation and differentiation of nerves and other cell types. Neurotransmitter receptors exist as different subtypes which are differentially expressed in various tissues and neurotransmitters such as acetylcholine evoke responses throughout the central and peripheral nervous systems. The muscarinic acetylcholine receptors play important roles in a variety of complex neural activities such as learning, memory, arousal and motor and sensory modulation. These receptors have also been implicated in several central nervous system disorders such as Alzheimer's disease, Parkinson's disease, depression and schizophrenia.

Some agents that are involved in a signal transduction pathway regulating one ion channel, for example a potassium channel, may also be involved in one or more other pathways regulating one or more other ion channels, for example a calcium channel. Dolphin, Ann. Rev. Physiol., 52:243-55 (1990); Wilk-Blaszczak et al., Neuron, 12:109-116 (1994). Ion channels may be regulated either with or without a cytosolic second messenger. Hille, Neuron, 9:187-195 (1992). One possible cytosolic second messenger is a tyrosine kinase. Huang et al., Cell, 75:1145-1156 (1993), incorporated herein by reference in its entirety, including any drawings.

The receptors involved in the signal transduction pathways that regulate ion channels are ultimately linked to the ion channels by various intermediate events and agents. For example, such events include an increase in intracellular calcium and inositol triphosphate and production of endothelin. Frucht, et al., Cancer Research, 52:1114-1122 (1992); Schrey, et al., Cancer Research, 52:1786-1790 (1992). Intermediary agents include bombesin, which stimulates DNA synthesis and the phosphorylation of a specific protein kinase C substrate. Rodriguez-Pena, et al., Biochemical and Biophysical Research Communication, 140(1):379-385 (1986); Fisher and Schonbrunn, The Journal of Biological Chemistry, 263(6):2208-2816 (1988).

### SUMMARY OF THE INVENTION

The present invention relates to PYK2 polypeptides, nucleic acids encoding such polypeptides, cells, tissues and animals containing such polypeptides and nucleic acids, antibodies to such polypeptides, assays utilizing such polypeptides, and methods relating to all of the foregoing. PYK2 polypeptides are involved in various signal transduction pathways and thus the present invention provides several agents and methods useful for diagnosing, treating, and preventing various diseases or conditions associated with abnormalities in these pathways.

The present invention is based in part upon the identification and isolation of a novel non-receptor tyrosine kinase, termed PYK2. Without wishing to be bound to any particular theory, it appears that PYK2 participates in at least two signal transduction pathways.

The first signal transduction pathway is activated when such extracellular signals as bradykinin or acetylcholine bind G protein-coupled receptor protein kinases. Receptor protein kinases that are G protein coupled include, but are not limited to, Lyn and Syk which are located in the B-cells of the immune system.

Another PYK2 signal transduction pathway is activiated when growth factors bind receptor protein kinases. The receptors dimerize and then cross phosphorylate one another. The phosphate moieties now attached to the receptor protein kinases attract other signalling molecules to these receptors located at the plasma membrane. Such signalling molecules can be, among others, sos, shc, or grb. Complexes such as an EGFR/grb-2/sos complex can activate ras molecules also located at the plasma membrane. Ras molecules can then activate signalling molecules that are not attached to the plasma membrane. These cytsolic signalling molecules can propogate an extracellular signal to the nucleus and promote the production of cellular agents necessary for a response to the extracellular signal. Examples of the cytosolic signalling molecules are proteins involved in the MAP kinase signalling cascade.

The description provided herein indicates that PYK2 may combine the G protein-coupled pathway with the sos/grb pathway for MAP kinase signal transduction activation in repsonse to stimulation by G protein-coupled receptors. The invention also indicates that PYK2 brings these two pathways together by binding src, another protein kinase involved in signal transduction events. Thus, the invention provides new targets for therapeutics effective for treating cell proliferative diseases such as cancer and/or cell differentiation disorders.

PYK2 has a predicted molecular weight of 111 kD and contains five domains: (1) a relatively long N-terminal domain from amino acid 1 to amino acid 417; (2) a kinase catalytic domain from amino acid 418 to amino acid 679 (contains nucleotide binding domain at amino acid 431 to amino acid 439 and an ATP binding site at amino acid 457); (3) a proline rich domain from amino acid 713 to amino acid 733; (4) another proline rich domain from amino acid 843 to amino acid 860; and (5) a C-terminal focal adhesion targeting (FAT) domain from amino acid 861 to amino acid 1009. PYK2 does not contain a SH2 or SH3 domain. Amino acids 696-1009 of PYK2 show homology to FRWK, the c-terminal fragments of FAK. Other features of the PYK2 sequence include the following: (1) amino acid 402 is the major autophophorylation site of PYK2 and is a Src SH2 binding site; (2) amino acid 599 is an autophosphorylation site in an activation loop of PYK2 kinase; (3) amino acid 881 is an autophosphorylation site and a GRB2 binding site; and (4) amino acid 906 is an auto phosphorylation site and SHP-2 (PTP-10) binding site.

The FAT domain of PYK2 has about 62% similarity to the FAT domain of another non-receptor tyrosine kinase, FAK, which is also activated by G-coupled proteins. The overall similarity between PYK2 and FAK is about 52%. PYK2 is expressed principally in neural tissues, although expression can also be detected in hematopoietic cells at early stages of development and in some tumor cell lines. The expression of PYK2 does not correspond with the expression of FAK.

PYK2 is believed to regulate the activity of potassium channels in response to neurotransmitter signalling. PYK2 enzymatic activity is positively regulated by phosphorylation on tyrosine and results in response to binding of bradykinin, TPA, calcium ionophore, carbachol, TPA+forskolin, and membrane depolarization. The combination of toxins known to positively regulate G-coupled receptor signalling (such as pertusis toxin, cholera toxins, TPA and bradykinin) increases the phosphorylation of PYK2.

Activated PYK2 phosphorylates RAK, a delayed rectifier type potassium channel, and thus suppresses RAK activity. In the same system, FAK does not phosphorylate RAK. PYK2 is responsible for regulating neurotransmitter signalling and thus may be used to treat conditions of nervous system by enhancing or inhibiting such signalling.

Thus, in a first aspect the invention features an isolated, purified, enriched or recombinant nucleic acid encoding a PYK2 polypeptide.

In preferred embodiments the isolated nucleic acid comprises, consists essentially of, or consists of a nucleic acid sequence set forth in the full length nucleic acid sequence SEQ ID NO:1 or at least 27, 30, 35, 40 or 50 contiguous nucleotides thereof and the PYK2 polypeptide comprises, consists essentially of, or consists of at least 9, 10, 15, 20, or 30 contiguous amino acids of a PYK2 polypeptide.

Compositions and probes of the present invention may contain human nucleic acid encoding a PYK-2 polypeptide but are substantially free of nucleic acid not encoding a human PYK-2 polypeptide. The human nucleic acid encoding a PYK-2 polypeptide is at least 18 contiguous bases of the nucleotide sequence set forth in SEQ. ID NO. 1 and will selectively hybridize to human genomic DNA encoding a PYK-2 polypeptide, or is complementary to such a sequence. The nucleic acid may be isolated from a natural source by cDNA cloning or subtractive hybridization; the natural source may be blood, semen, and tissue of various organisms including eukaryotes, mammals, birds, fish, plants, gorillas, rhesus monkeys, chimpanzees and humans; and the nucleic acid may be synthesized by the triester method or by using an automated DNA synthesizer. In yet other preferred embodiments the nucleic acid is a conserved or unique region, for example those useful for the design of hybridization probes to facilitate identification and cloning of additional polypeptides, the design of PCR probes to facilitate cloning of additional polypeptides, and obtaining antibodies to polypeptide regions.

The invention also features a nucleic acid probe for the detection of a PYK2 polypeptide or nucleic acid encoding a PYK2 polypeptide in a sample. The nucleic acid probe contains nucleic acid that will hybridize to a sequence set forth in SEQ ID NO:1.

In preferred embodiments the nucleic acid probe hybridizes to nucleic acid encoding at least 12, 27, 30, 35, 40 or 50 contiguous amino acids of the full-length sequence set forth in SEQ ID NO:2. Various low or high stringency hybridization conditions may be used depending upon the specificity and selectivity desired.

Methods for using the probes include detecting the presence or amount PYK2 RNA in a sample by contacting the sample with a nucleic acid probe under conditions such that hybridization occurs and detecting the presence or amount of the probe bound to PYK2 RNA. The nucleic acid duplex formed between the probe and a nucleic acid sequence coding for a PYK2 polypeptide may be used in the identification of the sequence of the nucleic acid detected (for example see, Nelson et al., in Nonisotopic DNA Probe Techniques, p. 275 Academic Press, San Diego (Kricka, ed., 1992) hereby incorporated by reference herein in its entirety, including any drawings). Kits for performing such methods may be constructed to include a container means having disposed therein a nucleic acid probe.

The invention also features recombinant nucleic acid, preferably in a cell or an organism. The recombinant nucleic acid may contain a sequence set forth in SEQ ID NO:1 and a vector or a promoter effective to initiate transcription in a host cell. The recombinant nucleic acid can alternatively contain a transcriptional initiation region functional in a cell, a sequence complimentary to an RNA sequence encoding a PYK2 polypeptide and a transcriptional termination region functional in a cell.

In another aspect the invention features an isolated, enriched or purified PYK2 polypeptide.

In preferred embodiments the FYK-2 polypeptide contains at least 9, 10, 15, 20, or 30 contiguous amino acids of the full-length sequence set forth in SEQ ID NO:2.

In yet another aspect the invention features a purified antibody (e.g., a monoclonal or polyclonal antibody) having specific binding affinity to a PYK2 polypeptide. The antibody contains a sequence of amino acids that is able to specifically bind to a PYK2 polypeptide.

Antibodies having specific binding affinity to a PYK2 polypeptide may be used in methods for detecting the presence and/or amount of a PYK2 polypeptide is a sample by contacting the sample with the antibody under conditions such that an immunocomplex forms and detecting the presence and/or amount of the antibody conjugated to the PYK2 polypeptide. Diagnostic kits for performing such methods may be constructed to include a first container means containing the antibody and a second container means having a conjugate of a binding partner of the antibody and a label.

In another aspect the invention features a hybridoma which produces an antibody having specific binding affinity to a PYK2 polypeptide.

In preferred embodiments the PYK2 antibody comprises a sequence of amino acids that is able to specifically bind a PYK2 polypeptide.

Another aspect of the invention features a method of detecting the presence or amount of a compound capable of binding to a PYK2 polypeptide. The method involves incubating the compound with a PYK2 polypeptide and detecting the presence or amount of the compound bound to the PYK2 polypeptide.

In preferred embodiments, the compound inhibits a phosphorylation activity of PYK2 and is selected from the group consisting of tyrphostins, quinazolines, quinaxolines, and quinolines. The present invention also features compounds capable of binding and inhibiting PYK2 polypeptide that are identified by methods described above.

In another aspect the invention features a method of screening potential agents useful for treatment of a disease or condition characterized by an abnormality in a signal transduction pathway that contains an interaction between a PYK2 polypeptide and a natural binding partner (NBP). The method involves assaying potential agents for those able to promote or disrupt the interaction as an indication of a useful agent.

Specific diseases or disorders which might be treated or prevented, based upon the affected cells include: myasthenia gravis; neuroblastoma; disorders caused by neuronal toxins such as cholera toxin, pertusis toxin, or snake venom; acute megakaryocytic myelosis; thrombocytopenia; those of the central nervous system such as seizures, stroke, head trauma, spinal cord injury, hypoxia-induced nerve cell damage such as in cardiac arrest or neonatal distress, epilepsy, neurodegenerative diseases such as Alzheimer's disease, Huntington's disease and Parkinson's disease, dementia, muscle tension, depression, anxiety, panic disorder, obsessive-compulsive disorder, post-traumatic stress disorder, schizophrenia, neuroleptic malignant syndrome, and Tourette's syndrome. Conditions that may be treated by PYK2 inhibitors include epilepsy, schizophrenia, extreme hyperactivity in children, chronic pain, and acute pain. Examples of conditions that may be treated by PYK2 enhancers (for example a phosphatase inhibitor) include stroke, Alzheimer's, Parkinson's, other neurodegenerative diseases and migraine.

Preferred disorders include epilepsy, stroke, schizophrenia, and Parkinson's disorder, as there is a well established relationship between these disorders and the function of potassium channels.

In preferred embodiments, the methods described herein involve identifying a patient in need of treatment. Those skilled in the art will recognize that various techniques may be used to identify such patients. For example, cellular potassium levels may be measured or the individuals genes may be examined for a defect.

In preferred embodiments the screening method involves growing cells (i.e., in a dish) that either naturally or recombinantly express a G-coupled protein receptor, PYK2, and RAK. The test compound is added at a concentration from 0.1 uM to 100 uM and the mixture is incubated from 5 minutes to 2 hours. The ligand is added to the G-coupled protein receptor for preferably 5 to 30 minutes and the cells are lysed. RAK is isolated using immunoprecipitation or ELISA by binding to a specific monoclonal antibody. The amount of phosphorylation compared to cells that were not exposed to a test compound is measured using an anti-phosphotyrosine antibody (preferably polyclonal). Examples of compounds that could be tested in such screening methods include tyrphostins, quinazolines, quinoxolines, and quinolines.

The quinazolines, tyrphostins, quinolines, and quinoxolines referred to above include well known compounds such as those described in the literature. For example, representative publications describing quinazoline include Barker et al., EPO Publication No. 0 520 722 A1; Jones et al., U.S. Pat. No. 4,447,608; Kabbe et al., U.S. Pat. No. 4,757,072; Kaul and Vougioukas, U.S. Pat. No. 5, 316,553; Kreighbaum and Comer, U.S. Pat. No. 4,343,940; Pegg and Wardleworth, EPO Publication No. 0 562 734 A1; Barker et al., Proc. of Am. Assoc. for Cancer Research 32:327 (1991); Bertino, J. R., Cancer Research 3:293-304 (1979); Bertino, J. R., Cancer Research 9(2 part 1):293-304 (1979); Curtin et al., Br. J. Cancer 53:361-368 (1986); Fernandes et al., Cancer Research 43:1117-1123 (1983); Ferris et al. J. Org. Chem. 44(2):173-178; Fry et al., Science 265:1093-1095 (1994); Jackman et al., Cancer Research 51:5579-5586 (1981); Jones et al. J. Med. Chem. 29(6):1114-1118; Lee and Skibo, Biochemistry 26(23):7355-7362 (1987); Lemus et al., J. Org. Chem. 54:3511-3518 (1989); Ley and Seng, Synthesis 1975:415-522 (1975); Maxwell et al., Magnetic Resonance in Medicine 17:189-196 (1991); Mini et al., Cancer Research 45:325-330 (1985); Phillips and Castle, J. Heterocyclic Chem. 17(19):1489-1596 (1980); Reece et al., Cancer Research 47(11):2996-2999 (1977); Sculier et al., Cancer Immunol. and Immunother. 23:A65 (1986); Sikora et al., Cancer Letters 23:289-295 (1984); Sikora et al., Analytical Biochem. 172:344-355 (1988); all of which are incorporated herein by reference in their entirety, including any drawings.

Quinoxaline is described in Kaul and Vougioukas, U.S. Pat. No. 5,316,553, incorporated herein by reference in its entirety, including any drawings.

Quinolines are described in Dolle et al., J. Med. Chem. 37:2627-2629 (1994); MaGuire, J. Med. Chem. 37:2129-2131 (1994); Burke et al., J. Med. Chem. 36:425-432 (1993); and Burke et al. BioOrganic Med. Chem. Letters 2:1771-1774 (1992), all of which are incorporated by reference in their entirety, including any drawings.

Tyrphostins are described in Allen et al., Clin. Exp. Immunol. 91:141-156 (1993); Anafi et al., Blood 82:12:3524-3529 (1993); Baker et al., J. Cell Sci. 102:543-555 (1992); Bilder et al., Amer. Physiol. Soc. pp. 6363-6143:C721-C730 (1991); Brunton et al., Proceedings of Amer. Assoc. Cancer Rsch. 33:558 (1992); Bryckaert et al., Experimental Cell Research 199:255-261 (1992); Dong et al., J. Leukocyte Biology 53:53-60 (1993); Dong et al., J. Immunol. 151(5):2717-2724 (1993); Gazit et al., J. Med. Chem. 32:2344-2352 (1989); Gazit et al., “J. Med. Chem. 36:3556-3564 (1993); Kaur et al., Anti-Cancer Drugs 5:213-222 (1994); Kaur et al., King et al., Biochem. J. 275:413-418 (1991); Kuo et al., Cancer Letters 74:197-202 (1993); Levitzki, A., The FASEB J. 6:3275-3282 (1992); Lyall et al., J. Biol. Chem. 264:14503-14509 (1989); Peterson et al., The Prostate 22:335-345 (1993); Pillemer et al., Int. J. Cancer 50:80-85 (1992); Posner et al., Molecular Pharmacology 45:673-683 (1993); Rendu et al., Biol. Pharmacology 44(5):881-888 (1992); Sauro and Thomas, Life Sciences 53:371-376 (1993); Sauro and Thomas, J. Pharm. and Experimental Therapeutics 267(3):119-1125 (1993); Wolbring et al., J. Biol. Chem. 269(36):22470-22472 (1994); and Yoneda et al., Cancer Research 51:4430-4435 (1991); all of which are incorporated herein by reference in their entirety, including any drawings.

Other compounds that could be tested in such screening methods include oxindolinones such as those described in U.S. patent application Ser. No. 08/702,232 filed Aug. 23, 1996, incorporated herein by reference in its entirety, including any drawings.

In another aspect the invention features a method of diagnosis of an organism for a disease or condition characterized by an abnormality in a signal transduction pathway that contains an interaction between a PYK2 polypeptide and a NBP. The method involves detecting the level of interaction as an indication of said disease or condition.

Yet another aspect of the invention features a method for treatment of an organism having a disease or condition characterized by an abnormality in a signal transduction pathway. The signal transduction pathway contains an interaction between a PYK2 polypeptide and a NBP and the method involves promoting or disrupting the interaction, including methods that target the PYK2:NBP interaction directly, as well as methods that target other points along the pathway.

In preferred embodiments the signal transduction pathway regulates an ion channel, for example, a potassium ion, the disease or condition which is diagnosed or treated are those described above, the agent is a dominant negative mutant protein provided by gene therapy or other equivalent methods as described below and the agents is therapeutically effective and has an EC₅₀ or IC₅₀ as described below.

An EC₅₀ or IC₅₀ of less than or equal to 100 μM is preferable, and even more preferably less than or equal to 50 μM, and most preferably less that or equal to 20 μM. Such lower EC₅₀'s or IC₅₀'s are advantageous since they allow lower concentrations of molecules to be used in vivo or in vitro for therapy or diagnosis. The discovery of molecules with such low EC₅₀'s and IC₅₀'s enables the design and synthesis of additional molecules having similar potency and effectiveness. In addition, the molecule may have an EC₅₀ or IC₅₀ less than or equal to 100 μM at one or more, but not all cells chosen from the group consisting of parathyroid cell, bone osteoclast, juxtaglomerular kidney cell, proximal tubule kidney cell, distal tubule kidney cell, cell of the thick ascending limb of Henle's loop and/or collecting duct, central nervous system cell, keratinocyte in the epidermis, parafollicular cell in the thyroid (C-cell), intestinal cell, trophoblast in the placenta, platelet, vascular smooth muscle cell, cardiac atrial cell, gastrin-secreting cell, glucagon-secreting cell, kidney mesangial cell, mammary cell, beta cell, fat/adipose cell, immune cell and GI tract cell.

In other aspects, the invention provides transgenic, nonhuman mammals containing a transgene encoding a PYK2 polypeptide or a gene effecting the expression of a PYK2 polypeptide. Such transgenic nonhuman mammals are particularly useful as an in vivo test system for studying the effects of introducing a PYK2 polypeptide, regulating the expression of a PYK2 polypeptide (i.e., through the introduction of additional genes, antisense nucleic acids, or ribozymes).

In another aspect, the invention describes a polypeptide comprising a recombinant PYK2 polypeptide or a unique fragment thereof. By “unique fragment,” is meant an amino acid sequence present in a full-length PYK2 polypeptide that is not present in any other naturally occurring polypeptide. Preferably, such a sequence comprises 6 contiguous amino acids present in the full sequence. More preferably, such a sequence comprises 12 contiguous amino acids present in the full sequence. Even more preferably, such a sequence comprises 18 contiguous amino acids present in the full sequence.

In another aspect, the invention describes a recombinant cell or tissue containing a purified nucleic acid coding for a PYK2 polypeptide. In such cells, the nucleic acid may be under the control of its genomic regulatory elements, or may be under the control of exogenous regulatory elements including an exogenous promoter. By “exogenous” it is meant a promoter that is not normally coupled in vivo transcriptionally to the coding sequence for the PYK2 polypeptide.

In another aspect, the invention features a PYK2 polypeptide binding agent able to bind to a PYK2 polypeptide. The binding agent is preferably a purified antibody which recognizes an epitope present on a PYK2 polypeptide. Other binding agents include molecules which bind to the PYK2 polypeptide and analogous molecules which bind to a PYK2 polypeptide.

Other features and advantages of the invention will be apparent from the following description of the preferred embodiments thereof, and from the claims.

### BRIEF DESCRIPTION OF THE FIGURES AND TABLES

FIG. 1 shows a schematic representation of the PYK2 domains (including a kinase domain, a proline rich domain, and a Fat domain) and potential binding sites (including YAEI, YLNV, and YVVV).

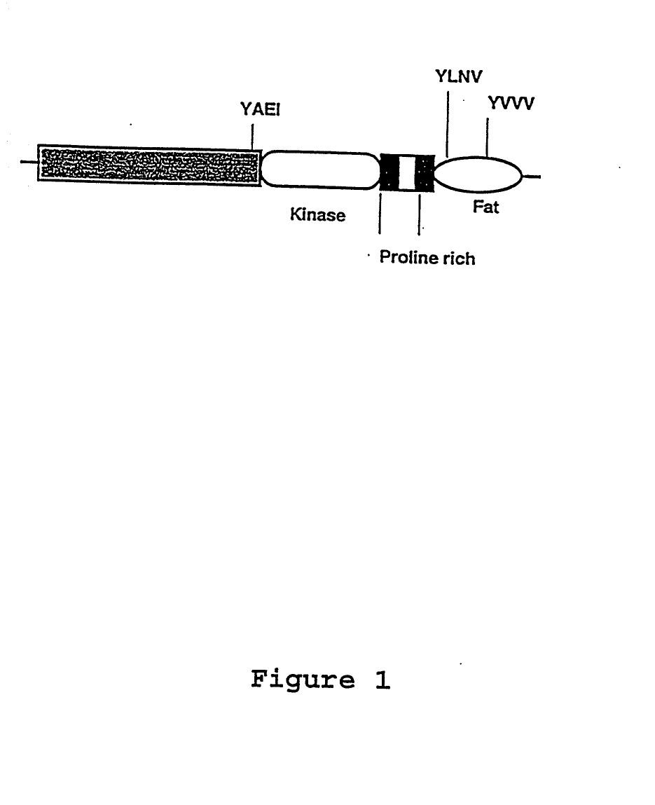

FIG. 2 shows a possible mechanism for the membrane depolarization and calcium influx that stimulate MEK and MAP kinase via activation of Ras. In PC12 cells, membrane depolarization leads to calcium influx through L-type calcium channels and activates MAP kinase. Calcium influx leads to activation of Ras and the activation of MAP in response to calcium influx is inhibited by a dominant negative mutant of Ras. Elevation of intracellular calcium concentration by various stimuli leads to the activation of PYK2. PYK2 recruits Shc/Grb2/Sos complex leading to the activation of a signaling, pathway composed of Ras, Raf, MAPKK, MAPK to relay signals to the cell nucleus.

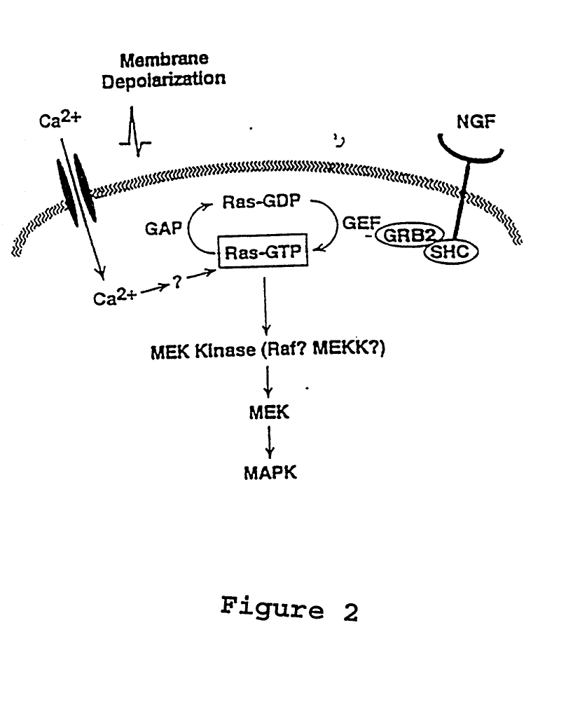

FIG. 3 shows a model for the extracellular stimuli that activate PYK2 and potential target molecule that is tyrosine phosphorylated in response to PYK2 activation. The tyrosine kinase activity of PYK2 is activated by a variety of extracellular signals that stimulate calcium influx including activation of the nicotnic acetylcholine receptor by carbachol, membrane depolarzation by KCl (75 mM), and treatment with a calcium ionophore. Activation of PYK2 by these stimuli requires the presence of extracellular calcium. PYK2 is also stimulated in response to bradykinin (BK) induced activation of its G-protein coupled receptor leading, to PI hydrolysis and Ca⁺² release from internal stores. PYK2 is also activated in response to phorbol ester (PMA) treatment that binds to and activates several PKC isozymes. Co-expression experiments in transfected cells and in frog oocytes show that activation of PYK2 leads to tyrosine phosphorylation (thick arrow) of the delayed rectifier-type K⁺ channel Kv1.2 and to suppression of Kv1.2 channel mediated currents.

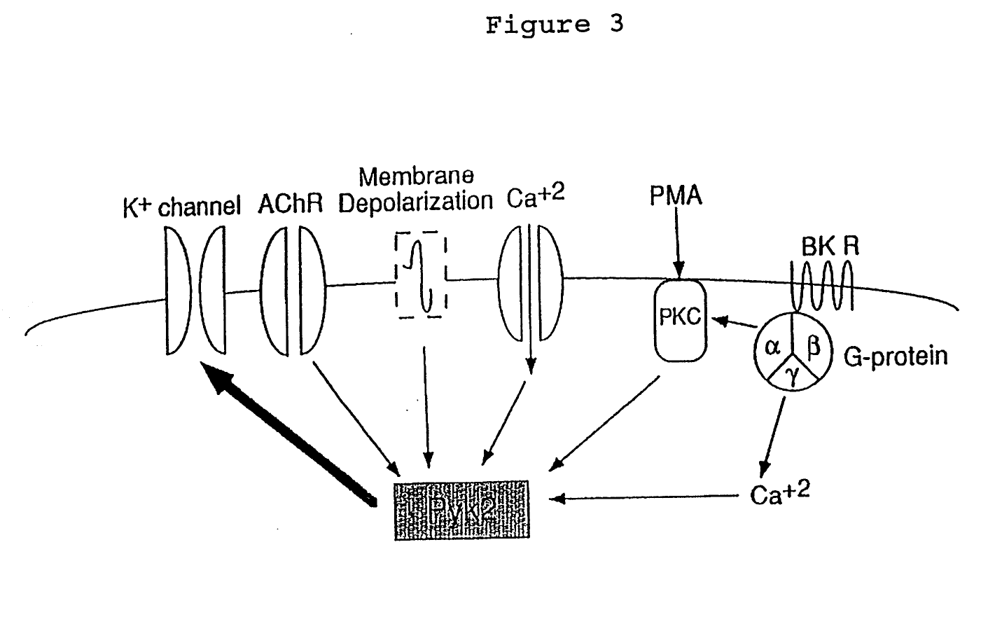

FIG. 4 shows an alignment of PYK-2 amino acids to those of 4 other proteins, Fak, Fer, HER4 and AB1.

Table 1 shows the expression pattern and levels of PYK2 in various cell lines as checked by multiple methods.

### DESCRIPTION OF THE PREFERRED EMBODIMENTS

The present invention relates to PYK2 polypeptides, nucleic acids encoding such polypeptides, cells, tissues and animals containing such nucleic acids, antibodies to such polypeptides, assays utilizing such polypeptides, and methods relating to all of the foregoing. Those skilled in the art will recognize that many of the methods described below in relation to PYK-2, a NBP, or a complex of PYK-2 and a NBP could also be utilized with respect to the other members of this group.

We describe the isolation and characterization of a novel non-receptor tyrosine kinase termed PYK2, that is highly expressed in the nervous system and in the adult rat brain. PYK2 is a second member of Fak family of non-receptor protein tyrosine kinases. However, PYK2 exhibits diffuse cytoplasmic localization unlike the preferential localization of Fak in focal adhesion areas.

The examples presented herein reveal a novel mechanism for the coupling, between G-protein coupled receptors and the MAP kinase signaling pathway. We also show that calcium influx induced by membrane depolorization following activation of the nicotinic acetylcholine receptor or other stimuli that cause calcium influx lead to the activation of PYK2, tyrosine phosphorylation of Shc, recruitment of Grb2/Sos and activation of the MAP kinase signaling pathway.

PYK2 is activated by extracellular signals that lead to calcium influx or calcium release from internal stores. PYK2 is phosphorylated on tyrosine residues in response to a variety of external stimuli that cause membrane depolarization and Ca⁺² influx such as the activation of the nicotinic acetylcholine receptor. Tyrosine phosphorylation of PYK2 is also stimulated by the neuropeptide bradykinin that activates a G-protein coupled receptor as well as by phorbol myristate acetate (PMA). Experiments in transfected cells and in Xenopus oocytes, microinjected with PYK2 mRNA, indicate that activation of PYK2 can lead to tyrosine phosphorylation of a delayed rectifier-type potassium channel protein and to suppression of potassium currents via this channel. These results suggest a novel mechanism by which a non-receptor tyrosine kinases, in the nervous system, can be both activated by and can modulate the action of ion-channel proteins.

Activation of PYK2 in PC12 cells by the same stimuli leads to the recruitment of Shc/Grb2/Sos complex and to the activation of the MAP kinase signaling pathway that relays signals to the cell nucleus. The experiments presented thus show that PYK2 may also provide a link between G protein coupled receptors and calcium influx and the MAP kinase signaling pathway; a pathway that relays signals from the cell surface to regulate transcriptional events in the nucleus. Overexpression of PYK2 leads to activation of MAP kinase. Moreover, the effects of PYK2 on tyrosine phosphorylation and action of the Kv1.2 potassium channel reveals a novel mechanism for heterologous regulation of ion-channel function by activation of an intermediate protein tyrosine kinase. PYK2 can, therefore, couple neuropeptide hormones that act via G-protein coupled receptors that stimulate phosphotydinositol hydrolysis and the action of target channel molecules.

Transient co-expression experiments of PYK2 with the delayed rectifier K+ channel Kv1.2 show that the channel protein undergoes tyrosine phosphorylation in response to PYK2 activation. Moreover, currents exhibited by Kv1.2 channel expressed in frog oocytes were blocked by co-expression of the PYK2 protein. However, co-expression of a kinase negative mutant of PYK2 released PMA induced suppression of Kv1.2 currents. PYK2 activation may provide a rapid and highly localized control mechanism for ion channel function and kinase activation induced by neuronal stimuli that elevate intracellular calcium leading, to neuronal integration and synaptic efficacy.

These results reveal a role for PYK2 in activation of the MAP kinase signaling pathway by ion channels, calcium influx and G-protein coupled receptors in PC12 cells and may provide a mechanism for signal transduction induced by these stimuli in the nervous system. Furthermore, tyrosine phosphorylation of Shc in response to membrane depolarization and carbachol treatment was dependent on the presence of extracellular calcium, indicating that calcium-influx plays a role in regulation of Shc phosphorylation by these stimuli.

Similarly, PYK2 may modulate the action of ion channels that mediate their responses via and are sensitive to intracellular calcium concentration. PYK2 may therefore provide an autoregulatory role for the very same channel responsible for PYK2 activation. A potential target of PYK2 is the nicotinic acetylcholine receptor. Activation of the nicotinic acetylcholine receptor in PC12 cells leads to strong and rapid tyrosine phosphorylation of PYK2.

The nicotinic acetylcholine receptor is subject to gylation can modulate the activity of the tyrosine phosphorylation. Tyrosine phosphorylation of Shc in response to carbachol treatment is induced via stimulation of the nicotinic acetylcholine receptor as determined by pharmacological analysis. The nicotinic agonist DMPP induced phosphorylation of Shc, whereas muscarine had no effect, the nicotinic antagonist mecamylamine blocked the effect of carbachol, whereas the muscarinic antagonist atropine had no effect. The effect of carbachol on tyrosine phosphoryiation of Shc was transient with maximum tyrosine phosphorylation detected after one minute followed by a rapid decline. NGF however, induced persistent stimulation of Shc phosphorylation for as long as five hours after the addition of NGF. The duration of Shc phosphorylation may have an important impact on the Ras signaling pathway and gene expression induced by these stimuli.

The model presented herein may represent the mechanism underlying calcium mediated regulation of gene expression in neuronal cells induced by MMDA receptor or voltage sensitive calcium channels. The expression pattern of PYK2, the external stimuli that activate this kinase together with its role in the control of MAP kinase signaling pathway suggests a potential role for PYK2 in the control of a broad array of processes in the central nervous system including neuronal plasticity. in the nervous svstem.

Since PYK2 activity is regulated by intracellular calcium level, both the temporal and spatial pattern of PYK2 activation, may represent a carbon copy or a replica of the spatial and temporal profile of intracellular calcium concentration. Calcium concentration inside cells is highly localized because of a variety of calcium binding proteins that provide a strong buffer. Moreover, in excitable cells the level of calcium can be regulated by voltage dependent calcium channels that induce large and transient increase in intracellular calcium concentration leading to calcium oscilations and calcium waves. PYK2 may provide a mechanism for rapid and highly localized control of ion channel function, as well as, localized activation of the MAP kinase signaling pathway.

Preliminary immunolocalization analysis indicates that PYK2 is expressed in hippocampal postsynaptic dendritic spines, suggesting a potential role of this kinase in synaptic plasticity mediated by calcium influx. Potassium channels are frequent targets for phosphorylation by tyrosine kinases that are activated by neurotransmitters or neuropeptides. Phosphorylation of other voltage gated channels or neurotransmitter receptors provides an important regulatory mechanism for modulation. Thus, PYK2 may represent an important coupling molecule between neuropeptides that activate G-protein coupled receptors or neurotransmitters that stimulate Ca+2 influx and downstream signaling events that reculate neuronal plasticity, cell excitability, and synaptic efficacy.

We have demonstrated that PYK2 is rapidly activated in response to a wide variety of extracellular stimuli. These stimuli include activation of an ion channel, stimulation of a G-protein coupled receptor, calcium influx following membrane depolarization as well as phorbol ester stimulation. Although the molecular mechanisms by which these signals induce the activation of PYK2 are not yet known, our results clearly show that elevation of intracellular calcium concentrations is crucial for PYK2 activation. The effect of PMA on PYK2 activation may indicate that PYK2 can be also activated by a PKC dependent pathway. The fact that PYK2 can be activated by an ion-channel, such as the nicotinic acetylcholine receptor, and by intracellular calcium raised the possibility that PYK2 may regulate ion-channel function by tyrosine phosphorylation.

We further analyzed agonist-induced MAP kinase activity in PC12 cell lines which stably overexpress a dominant interfering mutant of Grb2 lacking the N-terminal SH3 domain (Grb2 DN-SH3) or in PC12 cells which stably overexpress the proline rich tail of Sos (Sos-CT). Xie et al., J. Biol. Chem. 270, 30717-30724 (1995); Gishizky et al., Proc. Natl. Acad. Sci. USA 92, 10889-10893 (1995). Overexpression of Grb2 DN-SH3 in PC12 cells completely blocked LPA- or bradykinin-induced MAP kinase activation. Overexpression of Sos-CT strongly reduced MAP kinase activation in response to LPA and bradykinin stimulation. However, activation of PYK2 or Src was not affected by the dominant interfering mutants of Grb2 and Sos confirming that PYK2 and Src act upstream of Grb2 and Sos in the cascade of events leading to MAP kinase activation.

Experiments presented herein demonstrate that PYK2 can link both Gi- or Gq-protein coupled receptors with the MAP kinase signaling pathway in PC12 cells. Phosphorylation on Tyr402 of PYK2 leads to binding of the SH2 domain of Src and subsequent Src activation in response to either Gi- or Gq-protein coupled receptors. Overexpression of activated Src (Y527F) in PC12 cells induces tyrosine phosphorylation of PYK2, but does not stimulate PYK2 kinase activity.

It is possible therefore, that tyrosine phosphorylation of PYK2 is in part mediated by Src, thus generating docking sites for additional signaling proteins that are recruited by PYK2. We have demonstrated that activation of PYK2 leads to both direct recruitment of Grb2/Sos as well as indirect recruitment via tyrosine phosphorylation of Shc. We present experiments demonstrating that dominant interfering mutants of Grb2 or Sos confer strong inhibition on LPA- or bradykinin-induced activation of MAP kinase. These results are in accord with recent studies demonstrating that a Sos deletion mutant or a dominant interfering mutant of Shc blocked LPA- or thrombin-induced activation of MAP kinase respectively van Biesen et al., Nature 376:781-784 (1995); Chen et al., EMBO J. 15:1037-1044 (1996).

Taken together, these experiments underscore the central role of the Shc/Grb2/Sos complex in mediating MAP kinase activation not only by receptor tyrosine kinases, but also by Gi- and Gq-protein coupled receptors. Wan et al., Nature 380:541-544 (1996). In avian B-cells both Lyn and Syk are essential for activation of MAP kinase by Gi- and Gq-protein coupled receptors³. It appears therefore that a combination of different protein tyrosine kinases in different tissues and cell types may link G-protein coupled receptor with the MAP kinase signaling pathway. Src family protein tyrosine kinases, which are expressed in every cell type and tissue, appear to be a common and important component of this pathway, by acting together with cell-type specific protein tyrosine kinases such as PYK2 in PC12 cells or Syk in avian B-cells to bring about a cell-type specific signal for linking G-protein coupled receptors with MAP kinase signaling pathway and hence the transcriptional machinery.

Various other features and aspects of the invention are: nucleic acid molecules encoding a PYK2 polypeptide; nucleic acid probes for the detection of PYK2; a probe-based method and kit for detecting PYK2 messages in other organisms; DNA constructs comprising a PYK2 nucleic acid molecule and cells containing these constructs; purified PYK2 polypeptides; PYK2 antibodies and hybridomas; antibody-based methods and kits for detecting PYK2; identification of agents; isolation of compounds which interact with a PYK2 polypeptide; compositions of compounds that interact with PYK2 and PYK2 molecules; pharmaceutical formulations and modes of administration; derivatives of complexes; antibodies to complexes; disruption of PYK2 protein complexes; purification and production of complexes; transgenic animals containing PYK2 nucleic acid constructs; antisense and ribozyme approaches, gene therapy; and evaluation of disorders. Those skilled in the art appreciate that any modifications made to a complex can be manifested in a modification of any of the molecules in that complex. Thus, the invention includes any modifications to nucleic acid molecules, polypeptides, antibodies, or compounds in a complex. All of these aspects and features are explained in detail with respect to PYK-2 in PCT publication WO 96/18738, which is incorporated herein by reference in its entirety, including any drawings.

### EXAMPLES

The examples below are non-limiting and are merely representative of various aspects and features of the procedures used to identify the full-length nucleic and amino acid sequences of PYK-2. Experiments demonstrating PYK-2 expression, interaction and signalling activities are also provided.

Materials and Methods

Chemicals

Bradykinin, pertusis toxin, cholera toxin, forskolin, phorbol 12-myristate 13-acetate (PMA), calcium ionophore A23187, carbachol, muscarine, atrophine, mecamylamine, and 1,1-dimethyl-4-phenyl piperazinium iodide (DMPP) were purchased from Sigma.

Cloning of PYK2 cDNA

We have used the Grb2 adaptor protein as a specific probe for screening-expression libraries in order to isolate Grb2 binding proteins. One of the cloned proteins encoded a protein tyrosine kinase that contains a proline rich region that can bind in vitro to the SH3 domains of Grb2. This protein was termed PYK1 for proline rich tyrosine kinase 1. Comparison of the amino acid sequence of PYK1 to other tyrosine kinases, indicated that PYK1 is related to the Ack protein tyrosine kinase. Analysis of PYK1 sequence indicated that this kinase represents a new class of cytoplasmic protein tyrosine kinases.

In an attempt to isolate kinases related to PYK1, we applied the polymerase chain reaction (PCR) utilizing degenerate oligonucleotide primers, derived from PYK1 sequence according to the conserved motifs of the catalytic domains of PTKs. RNA from rat spinal cord was used to prepare cDNA utilizing the reverse transcriptase of Molony murine leukemia virus (^{BRL}) according to the manufacturer's protocol. The cDNA was amplified by PCR utilizing degenerate oligonucleotides primers corresponding to conserved tyrosine kinase motifs from subdomains TK6 and TK9 of PYK1; (the sense and antisense primers correspond to amino acid sequences IHRDLAARN [SEQ. ID NO 3] and WMFGVTLW [SEQ. ID NO 4] respectively). The PCR was carried out under the following conditions; 1 min at 94° C.; 1 min at 50° C. and 1 min at 68° C. for 35 cycles. PCR products were electrophoresed, checked by the size (−210 bp), purified and subcloned into pBluescript (Stratagene). Novel clones were screened by DNA sequencing. The cDNA insert of clone #38 was used as probe to screen human brain cDNA libraries (human fetal brain λgt 10 and human brain λgt 11, 6×10⁵ recombinant clones each) essentially as described by Maniatis ( ).

The complete amino acid sequence of a novel protein tyrosine kinase was isolated from human brain cDNA library and termed PYK2. The open reading frame of PYK2 encodes a protein of 1009 amino acids containing a long N-terminal sequence of 424 amino acids followed by a protein tyrosine kinase domain, two proline rich domains (29% and 23.3% proline respectively) and a large carboxy terminal region. The kinase domain of PYK2, contains several sequence motifs conserved among protein tyrosine kinases, including the tripeptide motif DFG, found in most kinases, and a consensus ATP binding motif GXGXXG followed by AXK sequence 17 amino acids residues downstream.

Comparison of the amino acid sequence of the kinase domain of PYK2 with other protein tyrosine kinases showed that the kinase core of PYK2 is most similar to the protein tyrosine kinase domains of Fak, Fer, Her4 and Ab1.

In addition to the sequence homology in the kinase domain, the flanking sequences and the overall structural organization of the PYK2 protein are similar to those of FAK indicating that PYK2 belong to the same family of non-receptor similar to those of Fak protein tyrosine kinases.

DNA Sequencing and Analysis

DNA sequencing was performed on both strands utilizing series of oligonucleotide primers and subclones. The nucleotide sequence and the deduced amino acid sequence were subjected to homology search with Genbank and PIR databases using FASTA and BLAST mail-server program.

Northern Blot Analysis

Total RNA was isolated from mouse tissues by the acid guanidinium thicynate-phenol-chloroform method (Anal. Biochem. 162; 156, 1987). Poly (A)⁺ RNA was denaturated with formaldehyde and electrophoresed on a 1% agarose/0.7% formaldehyde gel. RNAs were transferred to a nitrocellulose membrane and hybridized with ³²P-labeled probe that contained the cDNA insert of clone #38 as described above.

Antibodies

Antibodies against PYK2 were raised in rabbits immunized (HTI) either by GST fusion protein containing residues 362-647 or PYK2 or by synthetic peptide corresponding the 15 amino acids at the N-terminal end of PYK2. Antisera were checked by immunoprecipitation and immunoblot analysis, and the specificity was confirmed either by reactivity to the related protein Fak or by competition with the antigenic or control peptides.

Antibodies against PYK-2 were raised in rabbits immunized either with GST fusion protein containing residues of PYK-2 or with synthetic peptide correspondingly the 15 amino acids at the N-terminal end of PYK-2. The antibodies are specific to PYK-2 and they do not cross react with FAK.

Cells and Cell Culture

PC12-rat pheochromocytoma cells were cultured in Dulbecco's modified Eagle's medium containing 10% horse serum, 5% fetal bovine serum, 100 μg/ml streptomycin and 100 units of penicillin/ml. NIH3T3, 293, GP+E-86 and PA317 cells were grown in Dulbecco's modified Eagle's medium containing 10% fetal bovine serum, 100 μg/ml streptomycin and 100 unites of penicillin/ml.

Transfections and Infections

For stable expression in PC12 cells, PYK2 was subcloned into the retroviral vector PLXSN (Miller and Rosman, Biotechnigues 7:980, 1989). The construct was used to transfect GP+E-86 cells using lipofectimine reagent (GIBCO BRL). 48 hours after transfection, virus containing supernatants were collected. Pure retrovirus-containing cell-free supernatant were added to PC12 cells in the presence of polybrene (8 μg/ml, Aldrich) for 4 hours (MCB 12 491, 1992). After 24 hours, infected PC12 cells were split into growing medium containing 350 μl/mg G418. G418 resistant colonies were isolated two to three weeks later and the level of expression was determined by western blot analysis.

Stable cell lines of NIH3T3 that overexpress PYK2 were established by contransfection of PYK2 subcloned into PLSV together with pSV2neo utilizing lipofectamine reagent (GIBCO BRL). Following transfection the cells were grown in Dulbecco's modified Eagle's medium containing 10% fetal bovine serum and 1 mg/ml G418. Transient transfections into 293 cells were performed by using a calcium phosphate technique.

Constructs

GST-PYK2—a DNA fragment of λ900 bp corresponding to residues 362-647 of PYK2 was amplified by PCR utilizing the following oligonucleotide primers: 5′-CGGGATCCTCATCATCCATCCTAGGAAAGA-3′ (sense) [SEQ. ID NO 5] and 5′-CGGGAATTCGTCGTAGTCCCAGCAGCGGGT-3′ (antisense) [SEQ. ID NO 6].

The PCR product was digested with BamHI and EcoRI and subcloned into pGEX3X (Pharmacia). Expression of GST-PYK2 fusion protein was induced by the 1 mM IPTG essentially as described by Smith et al.,(Gene 67:31, 1988). The fusion protein was isolated by electroelution from SDS-PAGE.

PYK2—The full length cDNA sequence of PYK2 was subcloned into the following mammalian expression vectors: pLSV; downstream the SV40 early promoter, pLXSN-retroviral vector; downstream the Mo-MuLV long terminal repeat; pRK5; downstream the CMV promoter.

PYK2-HA—the influenza virus hemagglutinin peptide (YPYDVPDYAS) [SEQ. ID NO 7] was fused to the C-terminal end of PYK2 utilizing the following oligonucleotide primers in the PCR: 5′-CACAATGTCTTCAAACGCCAC-3¹ [SEQ. ID NO 8] and 5′-GGCTCTAGATCACGATGCGTAGTCAGGGACATCGTATGGGRACTCTGCAGGTGGGTGGGCCAG-3′. [SEQ. ID NO 9]. The amplified fragment was digested with RsrII and Xbal and used to substitute the corresponding fragment of PYK2. The nucleotide sequence of the final construct was confirmed by DNA sequencing.

Kinase negative mutant—in order to construct a kinase negative mutant, The Lys at position 457 was substituted to Ala by site directed mutagenesis (Clontech). The oligonucleotide sequence was designed to create a new restriction site of NruI. The nucleotide sequence of the mutant was confirmed by DNA sequencing. The oligonucleotide sequence that used for mutagenesis is: 5′-CAATGTAGCTGTCGCGACCTGCAAGAAAGAC-3′ [SEQ. ID NO 10] (Nrul site—bold, Lys-AAC substituted to Ala-GCG underline).

Rak-HA—The Rak cDNA was subcloned in pbluescript was obtained from Bernardu Rudi (NYU medical center). The influenza virus hemagglutinin peptide was fused to the C-terminal end of Rak essentially as described for PYK2. The oligonucleotide primers that were used in the PCR were: 5′-GCCAGCAGGCCATGTCACTGG-3′ [SEQ. ID NO 11] and 5′-CGGAATTCTTACGATGCGTAGTCAGGGACATCGTATGGGTAGACATCAGTTAACATTTTG-3′. [SEQ. ID NO 12] The PCR product was digested with BalI and EcoRI and was used to substitute the corresponding fragment at the C-terminal end of Rak. The Rak-HA cDNA was subcloned into pRK5 downstream the DMV promotor and into the retroviral vector pLXSN, downstream the Mo-MuLV long terminal repeat.

In vitro Mutagenesis

The mutagenic oligonucleotide (GAGTCAGACATCTTCGCAGAGATTCCC) SEQ ID NO: 26 and the trans oligonucleotide (GAATTCGATATCACGCGTGGCCGCCATGGC) SEQ ID NO: 27, were used to convert the tyrosine at position 402 to phenylalanine of PYK2 using a Clontech kit. Lev et al. Nature 376:737-745 (1995). The mutation was validated by DNA sequencing.

Immunoprecipitation and Immunoblot Analysis

Cells were lysed in lysis buffer containing 50 mM N-2-hydroxyethylpiperazine-N′-2-ethanesulferic acid (HEPES pH 7.5), 150 mM NaCl, 10% glycerol, 1% Triton X-100, 1.5 mM MgCl₂, 1 mM ethyleneglycol-bis (β-aminoethyl ether)-N,N,N′N′-tetraacetic acid (EGTA), 10 μg leupeptin per ml, 10 μg aprotinin per ml, 1 mM phenylmethylsulfonyl fluoride (PMSF), 200 μM sodium orthovanadate and 100 mM sodium fluoride. Immunoprecipitations were performed using protein A-sepharose (Pharmacia) coupled to specific antibodies. Immunoprecipitates were washed either with HNTG′ solution (20 mM HEPES buffer at pH 7.5, 150 mM NaCl, 10% glycerol, 0.1% Triton X-100, 100 mM sodium fluoride, 200 μM sodium orthovanadate) or successively with H′ solution (50 mM Tris-HCl pH8, 500 mM NaCl, 0.1% SDS, 0.2% Triton X-100, 100 mM NaF, 200AM sodium orthovanadate) and L′ solution (10 mM Tris-HCl pH 8, 0.1% Triton X-100, 100 mM NaF, 200 μM sodium orthovanadate).

The washed immunoprecipitates incubated for 5 min with gel sample buffer at 100° C. and analyzed by sodium dodecyl sulfate polyacrylamide gel electrophoresis (SDS-PAGE). In some experiments the gel-embedded proteins were electrophoretically transferred onto nitrocellulose. The blot was then blocked with TBS (10 mM Tris pH 7.4, 150 mM NaCl) that contained 5% low fat milk and 1% ovalbumin. Antisera or purified mAbs were then added in the same solution and incubation was carried out for 1 h at 22° C. For detection the filters were washed three times (5 min each wash) with TBS/0.05% Tween-20 and reacted for 45 min at room temperature with horseradish peroxidase-conjugated protein A. The enzyme was removed by washing as described above, and the filters were reacted for 1 min with a chemiluminescence reagent (ECL, Amersham) and exposed to an autoradiography film for 1-15 min.

In vitro Kinase Assay

This was carried out on immunoprecipitates in 50 μl HNTG (20 mM Hepes pH 7.5, 150 mM NaCl, 20% glycerol, 0.1% Triton X-100) containing 10 mM MnCl₂ and 5 μCi or [mN-³²P]ATP for 20 min at 22° C. The samples were washed with H′,M′ and, L′ washing solutions, boiled for 5 min in sample buffer and separated by SDS-PAGE.

Isolation of ACK/PYK

ACK/PYK may be isolated as described in Manser et al., Nature, 363:364-367, 1993. Comparison analysis of the full length sequence of ACK/PYK with other tyrosine kinases indicates that is not closely related to any of these, although it has some similarity to the focal adhesion kinase. Therefore, ACK/PYK represents a separate class of tyrosine kinases and isolation of related genes that belong to the same class is a major accomplishment.

### Example 1

### Isolation of PYK-2 cDNA

To identify genes related to the ACK/PYK protein tyrosine kinase, the polymerase chain reaction (PCR) was applied in combination with degenerated oligonucleotide primers based upon conserved motifs of the kinase domain of PTKs.

Oligonucleotides primers specifically designed to a highly conserved N-terminal motif of PTKs within subdomain TK6 (IHRDLAARN) SEQ ID NO 13. and ACK/PYK specific C-terminal primers within subdomain TK9 (WMFGVTLW) SEQ ID NO 14 were utilized. The amplification reactions with cDNA templates from 8 different sources gave rise to fragments of 0.2-0.9 kb. The PCR products were subcloned into pBlueScript and screened by DNA sequencing and hybridization under low stringency conditions.

A cDNA fragment of 210 bp from rat spinal cord was identified which is highly related to the Focal Adhesion Kinase (FAK). The fragment was sequenced in the 3′ and 5′ directions and was subsequently used as a probe to screen cDNA libraries (human fetal brain λgt 10 and human brain λgt 11, 6×10⁵ recombinant clones each).

Several overlapping clones spreading 1.5-3 kb were isolated and their cDNA inserts were analyzed by PCR, restriction mapping and sequencing. Two clones (#1 and #11) were chosen for further analysis and subcloning. Clone #1 contains an insert of 2.7 kb from the 5′ end of the gene, and clone #11 contains an insert of 3 kb from the 3′ end of the gene.

By utilizing a series of subclones and synthesized oligonucleotide primers the full length sequence of PYK-2 was determined. The sequence analysis resulted in a composite sequence of 3309 bp long which contains a 104 bp 5′ untranslated region, a 3021 bp coding region and 184 bp 3′ untranslated region. The ATG encoding the translation initiation codon is preceded by four translation stop codons in all reading frames.

The long open reading frame encodes a protein of 1007 amino acids (predicted molecular mass of 110,770 d) whose structural organization is very similar to FAK. The PYK-2 protein contains a long N-terminal sequence of 422 amino acids followed by a tyrosine kinase catalytic domain. The PYK-2 protein also contains the structural motifs common to all PTKs, two proline rich domains (19.6% and 17% proline respectively) and a focal adhesion targeting (FAT) motif in the C-terminal end. Comparison analysis of the amino acid sequence of PYK-2 with the human FAK revealed 52% identity between the two proteins. The kinase domain and the FAT sequence are most closely related (62% homology).

The PYK-2 protein contains several predicted binding sites for intracellular substrates. For example, YLMV [SEQ. ID NO 15] is a predicted binding site for GRB2 SH2 domain—tyrosine 879 of PYK-2. YVVV [SEQ. ID NO 16] is a predicted binding site for SHPTP2—tyrosine 903 of PYK-2. There are predicted phosphorylation sites for PKC, PKA and Ca/Calmodulin kinase. In addition, tyrosine 402 is a predicted autophosphorylation site of PYK-2 and it may be involved in the binding of a src SH2 domain. This is based on the homology between tyrosine 397 of FAK which was mapped as a major autophosphorylation site both in vivo and in vitro. This tyrosine provides an high affinity binding site for a src SH2 domain. Both tyrosine 397 of FAK and tyrosine 402 of PYK-2 are located at the juncture of the N-terminal and the catalytic domain and are followed by sequence (Y)AEI which is very similar to the consensus of the high affinity src SH2 domain binding peptide YEEI.

Total RNA from rat spinal cord was used to prepare cDNA utilizing the reverse transcriptase of Molony murine leukemia virus (‘Superscript’, BRL) according to the manufacturer's protocol. The cDNA was amplified by PCR utilizing degenerate oligonucleotides primers corresponding to conserved tyrosine kinase motifs from subdomains TK6 and TK9 of PYK1; (the sense and antisense primers correspond to amino acid sequences IHRDLAARN [SEQ. ID NO 17] and WMFGVTLW [SEQ. ID NO 18] respectively). The PCR was carried out under the following conditions; 1 min at 94° C.; 1 min at 50° C. and 1 min at 68° C. for 35 cycles. Amplified DNA was subcloned and sequence, resulting in identification of a novel tyrosine kinase termed PYK2. A λgt10 human fetal brain cDNA library (clontech) was screened with ³²P-labeled PCR clone corresponding to rat PYK2. Four overlapping clones were isolated, their DNA sequence was determined on both strands utilizing series of oligonucleotide primers. The 314-bp consensus sequence contains a single open reading frame of 3027 nucleotide preceded by a 105 nucleotide 5′-untranslated region. Amino acid sequence comparisons were performed using, the Smith-Waterman algorithm of MPSRCH (IntelliGenetic) on MasPar computer.

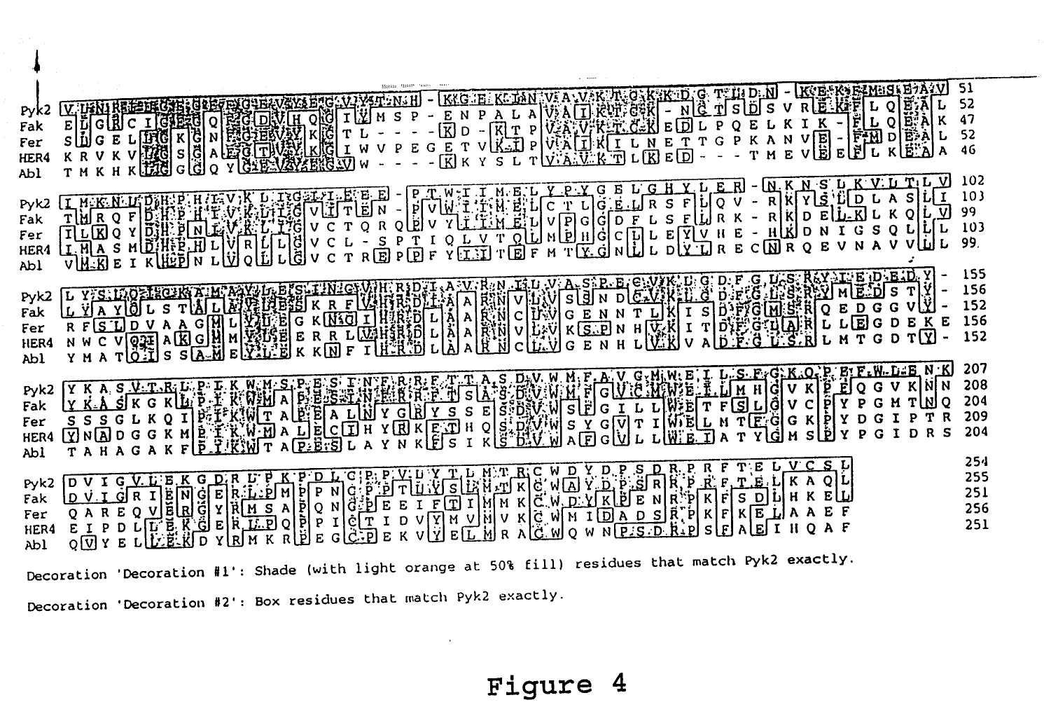

The deduced amino acid sequence of human PYK2 from cDNA clones is shown in FIG. 4. The tyrosine kinase domain is highlighted by a dark shaded box. Two proline-rich domains in the C-terminal region are boxed with light shading. Amino acid residues are numbered on the left. Comparison of the amino acid sequence of the catalytic domain of PYK2 with four human protein tyrosine kinases demonstrated 61%, 43%, 40% and 41% sequence identity between PYK2 and Fak, Fer, HER4 and Abl, respectively. The homology between PYK2 and Fak extends beyond the catalytic domain with 42% and 36% amino acid identify in the N-terminal and C-terminal regions, respectively.

### Example 2

### Pattern of PYK-2 Expression

PYK2 is highly expressed in the nervous system. We examined the expression pattern and the tissue distribution of PYK2 by Northern blot and by in situ hybridization analyses.

The tissue distribution of PYK-2 expression was determined by Northern blot analysis. Poly(A)⁺ RNAs were purified from mouse tissues (liver, lung, spleen, kidney, heart, brain, skin, uterus) and hybridized with two different probes corresponding to two different regions of the PYK-2 gene. The results were identical in both cases. A 4.2-4.5 kb PYK-2 transcript is relatively abundant in the brain but was also found in lower levels in the spleen and in the kidney.

Film autoradiography of a sagittal section through the adult rat brain shows very high levels of expression in the olfactory bulf (OB), hippocampus (Hi), and dentate gyrus (DG). Moderate levels of expression are seen in the cerebral cortex (Cx), striatum (S), and thalamus (T). Low levels of expression are seen in the cerebellum (Cb) and brainstem (BS).

Expression of PYK2 mRNA determined by Northern blot analysis of poly(A)+ from various human tissues. The northern blot was hybridized with 3.9 kb ³²P-labeled fragment containing the PYK2 cDNA in 50% foramide at 42° C.

Northern blot of mRNA isolated from various human brain sections (amygdala, caudate nucleus, corpus callosum, hippocampus, hypothalamus, substantia nigra, subthalamic nucleus and thalamus) revealed highest expression in the hippocampus and amygdala, moderate level of expression in the hypothalamus, thalamus and caudate nucleus and low level of expression in the corpus callosum and subthalamic nucleus.

These results are consistent with in situ hybridization analysis on day 7 post natal rat brain sections utilizing antisense probes derived from PYK2 sequence. The in situ hybridization analysis demonstrate that the olfactory bulb, the hippocampus and the dentate gyrus exhibit high level of PYK2 transcripts. Moderate levels of PYK2 expression was detected in the striatum, cerebral cortex and thalamus and low levels of expression was detected in the cerebellum and brainstem.

In order to characterize the PYK2 protein, NIH3T3 cells were transfected with a mammalian expression vector that encodes PYK2 protein with an influenza virus hemaglutanin peptide tag. PYK2 was immunoprecipicated with either anti-PYK2 or anti-HA antibodies from 3T3 transfected cell, whereas the endoaenous PYK2 protein was immunoprecipitated with anti-PYK2 antibodies from PC12 cells. These antibodies precipitated a protein that migrated in SDS gels with apparent molecular weight of 112 kDa. Addition of Y-[32p]ATP to immunoprecipitates from PYK2 transfected cells followed by SDS-PAGE analysis and autoradiography showed that PYK2 undergoes phosphorylation on tyrosine residues.

The expression of PYK-2 in different cell lines was analyzed by IP/IB utilizing anti-PYK-2 antibodies directed to the kinase domain as described previously (GST-PYK-2). The expression pattern is summarized in table 1. Some of the interesting observations are a mobility shift of PYK-2 after differentiation of CHRF and L8057 (premegakaryocyte cell lines) by TPA, high expression of Fak and PYK-2 in different cell lines; and in XC cells (rat sarcoma) PYK-2 is phosphorylated on tyrosine. PYK2 was immunoprecipitated from NIH3T3 cells, NIH3T3 cells that overexpress PYK2-HA and PC12 cells. The immunocomplexes were washed and resolved by 7.5% SDS-PAGE. immunoblotting was performed with anti-PYK2 antibodies. In vitro Kinase activity of PYK2. Cos cells were transiently transfected with PYK2-HA expression vector (+) or with an empty vector (−). The PYK2 protein was immunoprecipitated with anti-HA antibodies, the immunocomplexes were washed and subjected to in vitro kinase assay.

In situ hybridization was performed as follows: Fresh frozen rat brains were cut on a cryostat into 20-mm thick sections and thaw-mounted onto gelatin coated slides. The sections were fixed in 4% paraformaldehyde in 0.1 M sodium phosphate (pH=7.4) for 30 minutes and rinsed three times for 5 minutes each in PBS and one time for 10 minutes in 2× SSC. Two probes were used in the hybridization analysis, a 51 base oliconucleotide complementary to the sequence encoding amino acid 301-317, and a 51 base oligonucleotide complementary to the sequence encoding, amino acid 559-575 (from rat PCR product).

The oligonucleotides were labeled with a-³⁵S dATP (Du Pont-New England Nuclear) using terminal deoxynucleotidyl-transferase (Boehinger Mannheim) and purified using sephadex G-25 quick spin columns (Boehinger Mannheim). The specific activity of the labeled probes was between 5×10⁸ and 1×10⁹ cpm/mg. Prehybridization and hybridization were carried out in a buffer containing 50% deionized formamide, 4× SSC, 1× Denhardts' solution, 500 ug/ml denatured salmon sperm DNA, 250 ug/ml yeast tRNA, and 10% dextran sulfate. The tissue was incubated for 12 hours at 45° C. in hybridization solution containing the labeled probe (1×10⁶ cpm/section) and 10 mM dithlothreitol.

Controls for specificity were performed on adjacent sections by competitively inhibiting hybridization of the labeled olic,onucleotides with a 30-fold concentration of unlabeled oligonucleotide and by hybridization with sense probes. After hybridization, the sections were washed in two changes of 2× SSC at room temperature for 1 hour, 1× SCC at 55° C. for 30 minutes, 0.5× SSC at 55° C. for 30 minutes, and 0.5× SSC at room temperature for 15 minutes and then dehydrated in 60, 80, 95, and 100% ethanol. After air drying, the sections were exposed to x-ray film for 5 days. The sections were then dipped in Ilford K.5 photographic emulsion (Polysciences), exposed for 4 weeks at 4° C., and developed using Kodak D-19 developer and rapid fixer.

Emulsion autoradiography was examined by dark-field microscopy on a Zeiss axioskop. The influenza virus hemagglutinin peptide (YPYDVPDYAS) [SEQ. ID NO 19] tag was added to the C-terminal end of PYK2 utilizing the following oligonucleotide primers in the PCR: ′5-CACAATGTCTTCAAACGCCAC′3′[SEQ. ID NO 20] and ′5-GGCTCTAGATCACGATGCGTAGTCAGGGACATCGTATGGGTACTCTGCAGGTGGGT GGGCCAG-′3′ [SEQ. ID NO 21]. The amplified fragment was digested with RsrII and XbaI and used to substitute the corresponding fragment of PYK2. The nucleotide sequence of this construct was confirmed by DNA sequencing.

In vitro kinase assay was carried out on immunoprecipitates in 50 μl HNTG (20 mM Hepes pH 7.5, 150 mM NaCl, 20% glycerol, 0.1% Triton X-100) containing 10 mM MnCl₂ and 5 mCi of [λ-³²P] ATP for 20 min at 22° C. The samples were washed with H′(50 mim Tris-HCI pH 8, 500 mM NaCl, 0.1% SDS, 0.2% Triton X-100, 5 mM EGTA, 100 mM NaF, 200 μM sodium orthovanadate), M′ (50 mM Tris-HCl pH 8, 150 mM NaCl, 7.5 mM EDTA, 0.1% SDS, 0.2% Triton X-100, 100 mM NaF, 200 AM sodium orthovanadate) and L′ (10 mM Tris-HCl pH 8, 0.1% Triton X-100, 100 mM NaF, 200 μM sodium orthovanadate) washing, solutions, boiled for 5 min in sample buffer and separated by SDS-PAGE.

Antibodies against PYK2 were raised in rabbits immunized with GST fusion protein containing residues 62-647 of PYK-2. Antibodies against influenza virus hemagglutinin peptide) were purchased from Boehringer Mannheim. Cell lysis, immunoprecipitations and immunoblotting was performed essentially as described by Lev et al. Mol. Cell. Biol. 13, 2224-2234, 1993.

### Example 3

### Properties of PYK-2 Protein

In order to analyze the biochemical properties of PYK-2 the full length cDNA was subcloned into the two mammalian expression vectors RK5 and pLSV. In parallel, an expression vector encoding the PYK-2 protein fused to the influenza virus hemagglutinin peptide was constructed. This construct was used to identify the protein utilizing anti-HA antibodies.

pLSV-PYK-2-HA was transfected into cos cells. The protein was expressed at the predicted molecular mass (−116 kD) as determined by IP and IB with anti-HA antibodies. The protein is an active kinase as determined by in vitro kinase assay utilizing (λ³²P) ATP or an in vitro kinase assay utilizing cold ATP and immunoblotting with anti-phosphotyrosine antibodies.

The PYK-2 cDNA cloned in PLSV was cotransfected with pSV2neo into PC12 cells and NIH3T3 in order to establish stable cell lines. G418 resistant colonies were screened by immunoprecipitating and immunoblotting.

NIH3T3 cell lines were established that overexpress the PYK-2 and the PYK-2-HA protein. In these cells PYK-2 undergoes tyrosine phosphorylation in response to PDGF, EGF and aFGF. The level of phosphorylation is not so high. The stronger effect is achieved by TPA treatment (6 μM) after 15 min incubation at 37° C. as determined by time course analysis.

### Example 4

### Tyrosine Phosphorylation of PYK2 in Response to Carbachol, Membrane Depolarization and Ca+² Influx

The phosphorylation of PYK-2 on tyrosine residue in response to different stimulus was analyzed by immunoprecipitation of PYK-2 and immunoblotting with anti-phosphotyrosine antibodies and vice versa.

The following treatments were used: Bradykinin, TPA, forskolin, forskolin+TPA, bradykinin+forskolin, NGF, Neuropeptide Y, Cholera toxin, Cholera toxin+TPA, Cholera toxin+bradykinin, pertusis toxin, pertusis toxin+TPA, bradykinin+pertusis toxin, calcium ionophore A23187, bombesin.

The following results were obtained: PYK-2 undergoes tyrosine phosphorylation in response to TPA (1.6 μM 15 min at 37° C.), bradykinin (1 μM 1 min at 37° C.) and calcium ionophore A23187 (2 μM 15 min at 37° C.). Forskolin increase the response of TPA but does not give any signal by itself. Cholera toxin gave higher signal in combination with TPA and bradykinin but didn't cause phosphorylation of PYK-2 alone. Pertusis toxin also induced the response of TPA and bradykinin but didn't cause any response alone. In order to determined if the bradykinin effect is mediated by PKC signaling pathway attempts to down regulate PKC by chronic treatment with TPA (twice) did not give a clear answer.

One interpretation of these results is that PKC and PKA (and maybe Ca/calmudolin kinase) induce the autophosphorylation of PYK-2 in response to ser/the phosphorylation. This interpretation may be checked by utilizing specific inhibitors to PKC and PKA and by phosphamino-acid analysis.

Confluent PC12 cells in 150 mm plates were grown for 18 hours in DMEM containing 0.5% horse serum and 0.25% fetal bovine serum. The cells were sitmulated at 37° C. with different agonists as indicated. washed with cold PBS and lysed in 800 ml lysis buffer (Lev et al., supra).

The cell lysates were subjected to immunoprecipitation with anti-PYK2 antibodies. Following SDS-PAGE and transfer to nitrocellulose, the samples were immunoblotted with either antiphosphotyrosine (RC20, transduction laboratories) or anti-PYK2 antibodies.

Carbachol induces tyrosine phosphorylation of PYK2 via activation of the nicotinic acetylcholine receptor. Immunoprecipitates of PYK2 from PC12 cells that were subjected to the following treatments: muscarine (1 mM) or carbachol (1 mM) for 20 sec at 37° C. Carbachol(1 mM), DMPP (100 μM), or carbachol after pretreatment with the muscarinic antagonist atropine (100 nM) or the nicotinic antagonist mecamylamine (10 μM) for 5 min at 37° C. Incubation with carbachol in the presence or absence of EGTA (3 mM) as indicated. The immunocomplexes were resolved by SDS-PAGE, transferred to nitrocellulose, and probed with either anti-phosphotyrosine antibodies or with anti-PYK2 antibodies as indicated. Membrane depolarization and calcium ionophore induce tyrosine phosphorylation of PYK2. Immunoprecipitates of PYK2 from quiescent PC12 cells were subjected to the following treatments: incubation with 75 mM KCI in the presence or absence of EGTA (3 mM), incubation with 6 μM of the calcium ionophore A23187 for 15 min at 37° C. The immunoprecipitates were washed, resolved by 7.5% SDS-PAGE and immunoblotted with either antiphosphotyrosine antibodies or with anti-PYK2 antibodies.

Activation of PYK2 by carbachol, membrane depolarization and Ca+2 influx was studied. Since PYK2 is highly expressed in the central nervous system and in PC12 cells, we examined the effect of a variety of neuronal agonists on the phosphorylation state of PYK2. In these experiments, PC12 cells were treated with an agonist, lysed and subjected to immunoprecipitation with anti-PYK2 antibodies followed by SDS-PAGE analysis and immunoblotting with phosphotyrosine specific antibodies.

Stimulation of PC12 cells with carbachol induces strong, tyrosine phosphorylation of PYK2. We explored the possibility whether activation of both cholinergic receptor subtypes leads nicotinic and muscarinic receptors to tyrosine phosphorylation of PYK2. Pharmacological analysis with either subtype specific agonists, muscarine and DMPP or subtype specific antagonists, atropine and mecamylamine indicated that activation of PYK2 by carbachol is mediated via the nicotinic acetylcholine receptor. The phosphorylation of PYK2 in response to carbachol is very rapid; 5 second after applying carbachol to the cells, PYK2 became phosphorylated on tyrosine residues. Elimination of extracellular calcium by EGTA completely blocked agonist induced tyrosine phosphorylation of PYK2, indicating that calcium influx is required for carbachol induced PYK2 activation.

Stimulation of the nicotinic acetylcholine receptor induces membrane depolarization by cation influx via the ion channel pore. We have therefore checked whether membrane depolarization induced by a high concentration of potassium chloride will cause the same effect on PYK2 tyrosine phosphorylation. Depolarization of PC12 cells with 75 mM KCl induces rapid tyrosine phosphorylation of PYK2. The omission of calcium from the extracelluar medium completely abolished PYK2 tyrosine phosphorylation, indicating that activation of PYK2 is due to calcium influx rather than membrane depolarization per se. To further explore this possibility, we examined the effect of a calcium ionophore on PYK2 activation. PYK2 is phosphorylated on tyrosine residues following incubation with the calcium ionophore A23187. These results show that elevation of intracellular calcium in response to a variety of stimuli causes tyrosine phosphorylation of PYK2.

Tyrosine phosphoyriation of PYK2 in response to activation of a G protein coupled receptor was studied. We analyzed the effect of bradykinin on the phosphorylation state of PYK2. Bradykinin induces rapid tyrosine phosphorylation of PYK2 in PC12 cells. By contrast to stimulation of PYK2 phosphorylation in response to carbachol treatment or to membrane depolarization the effect of bradykinin was not influenced by the omission of extracellular calcium; bradykinin induced PYK2 phosphorylation in the absence of extracellular calcium or in the presence of EGTA.

Incubation of PC12 cells with phorbol myristate acetate (PMA) induced tyrosine phosphorylation of PYK2, suggesting that tyrosine phosphorylation of PYK2 could also be mediated via protein kinase C (PKC) activation. To determine whether bradykinin-induced phosphorylation of PYK2 is mediated via PKC, the cells were treated with bradykinin or PMA following down-regulation of PMA-sensitive PKC isozymes by prolonged treatment with PMA. Prolonged treatment with PMA completely abolished the effect of PMA, but had only a minor effect on bradykinin-stimulated tyrosine phosphorylation of PYK2. These results suggest that tyrosine phosphorylation of PYK2 can be induced by PKC-independent and by PKC-dependents mechanisms.

### Example 5

### Phosphorylation of RAK

293 cells in 65 mm plates were transiently transfected either with the potassium channel-RAK-HA alone, or together with Fak, PYK2 or the PYK2-kinase negative mutant (PKN). 12 hr following transfection the cells were grown in DMEM containing 0.3% fetal bovine serum for 24 hours. The cells were either stimulated with PMA (1.6 μM) or with calcium ionophore A23187 (6 μM) for 15 min at 37° C. or left unstimulated. The cells were solubilized and the expression level of each protein was determined by western blot analysis. The Rak protein was immunoprecipitated by anti-HA antibodies and its phosphorylation on tyrosine residues was analyzed by western blot analysis utilizing anti-phosphotyrosine antibodies following immunoprecipitation of the proteins either with anti-PYK2 antibodies (for PYK2 and PKN) or with anti Fax antibodies for (Fak).

The expression level of each protein (Rak PYK2, PKN and Fak) and the tyrosine phosphorylation of Rak, PYK2, PKN and Fak were measured.

Only the kinase active PYK2 protein phosphorylated the potassium channel. No phosphorylation was observed with kinase negative PYK2 or with FAK.

### Example 6

### Tyrosine Phosphorylation of PYK2 and Shc in Response to Activation of PC 12 Cells by Different Stimuli

PC12 cells were grown in DMEM containing 0.25% fetal bovine serum and 0.5% horse serum for 18 hours before stimulation. Following stimulation, the cells were washed with cold PBS and lysed in 0.8 ml lysis buffer (Lev et al., Mol. Cell. Biol. 13, 225-2234, 1993). PYK2 was immunoprecipitated by anti-PYK2 antibodies, the immunoprecipitates were resolved by 7.5% SDS-PAGE and immunoblotted either with anti-phosphotyrosine antibodies (RC20, transduction laboratories) or with anti-PYK2 antibodies. Antibodies against PYK2 were raised in rabbits.

Tyrosine phosphorylation of PYK2 in response to different stimuli was studied. Quiescent PC12 cells were stimulated at 37° C. with carbachol (1 mM, 20 sec), bradykinin (1 μM, 1 min KCI (75 mM, 3 min), PMA (1.6 μM, 15 min), A23I87 (6 μM, 15 min) or left unstimulated (−). PYK2 was immunoprecipitated from cell lysates with anti-PYK2 antibodies, followed by SDS-PAGE and immunoblotting, with antiphosphotyrosine or anti-PYK2 antibodies.

Tyrosine phosphorylation of Shc in response to bradykinin, carbachol, PMA and other stimuli was also measured. Quiescent PC12 cells were stimulated for 5 min at 37° C. with bradykinin (1 μM), carbachol (1 mM), KCl (75 mM), PMA (1.6 μM), NGF(100 ng/ml), or left unstimulated (−). The cells were also stimulated with carbachol (1 mM) or potassium chloride (75 mM) in the presence of 3 mM EGTA. Stimulations with DMPP (100 μM) or muscarine (1 mM) were preformed under the same conditions. Time-course of carbachol induced tyrosine phosphorylation of Shc was performed by incubation of the cells with 1 mM carbachol. The Shc proteins were immunoprecipitated with anti-Shc antibodies, the immunoprecipitates were resolved by SDS-PAGE (8%), transferred to nitrocellulose and immunoblotted with anti-phosphotyrosine antibodies.

### Example 7

### Association of PYK2 with Grb2 and Sos 1 in Intact Cells

In order to explore the possibility that calcium induced PYK2 activation is responsible for tyrosine phosphorylation of Shc and activation of the Ras/MAPK signaling pathway, we have examined the ability of PYK2 to recruit upstream regulatory elements of this signaling, pathway, such as Shc and Grb2. Human embryonic 293 cells were transiently transfected with different combinations of expression vectors that direct the synthesis of PYK2, a kinase negative PYK2 mutant (PKN) and the adaptor protein Grb2. The results show that Grb2 is directly associated with wild type PYK2 but not with the kinase negative mutant. Experiments with GST-fusion protein of Grb2 indicate that the association between Grb2 and PYK2 is mediated via its SH2 domain. Inspection of PYK2 primary structure shows that tyr881 is followed by a LNV sequence which was shown to be a canonical binding site for the SH2 domain of Grb2l9.

We next examined the interaction of PYK2 with the gauanine nucleotide releasing factor SOS I. Human embryonic kidney 293 cells were transfected with expression vectors encoding SOS 1, PYK2 and PKIN and subjected to immunoprecipitation/immunoblotting analysis with anti Sosl or anti-PYK2, antibodies, respectively. Wild type PYK2 but not the kinase negative mutant (PKN) was co-immunoprecipitated with the with Sosl protein. Hence, Grb2 is bound to Sosl via its SH3 domains and to PYK2 via its SH2 domain leadina to the recruitment of Sos by tyrosine phosphorylated PYK2.

Growth factor induced activation of receptor tyrosine kinases leads to a shift in the electrophoretic mobility of SOS protein. The mobility shift was shown to be due to phosphorylation by serine and thronine kinases which are dependent upon Ras activation including the MAP kinase 11, 12, 20. SOS I protein from PYKI− transfected cells exhibits reduced electrophoretic mobility as compared to SOS I protein. This experiment shows that PYK2 over-expression leads to the activation of the ser/thr kinases responsible for the phosphorylation of SOS 1.

293 cells were transiently transfected with the full length cDNAs of PYK2, PKN Grb2 and hSosl-HA cloned into the mammalian expression vectors pRK5 downstream to the CMV promotor, using the calcium phosphate precipitation method (Wigler et al., Cell 16, 777-785, 1979). Twelve hours after transfection, the cells were incubated in medium containing 0.2% fetal bovine serum for 24 hours. The cells were Ivsed, subjected to immunoprecipitation, resolved by SDS-PAGE (15% for Grb2 IPs, 7.5w for PYK2 IPs) and inununoblotted essentially as described (Lev et al., Mol. Cell Biol. 13, 2224-2234, 1993). For immunoblotting we used a mouse monoclonal antibody against Grb2 (Transduction laboratories #GI6720). The kinase negative mutant of PYK2 was constructed as described. A mammalian expression vector encodes the hSosl-HA was constructed as described (Aronheim et al., Cell 78, 949-961, 1994).

Embryonic human kidney 293 cells were transiently transfected with different combinations of mammalian expression vectors that direct the synthesis of Grb2, PYK2 and a kinase negative PYK2 point mutant (PKN). The cells were solubilized and immunoprecipitated with anti-Grb2, or anti-PYK2 antibodies. The immunocomplexes were washed, resolved by SDS-PAGE, transferred to nitrocellulose and immunoblotted with either anti-PYK2, or anti-Grb2 antibodies. The expression level of Grb2 in each cell line was determined by immunoblotting of total cell lysates with anti-Grb2 antibodies.

Embryonic human kidney 293 cells were transiently transfected with manunaiian expression vectors encoding hsosl-HA, hSosl-HA together with PYK2 or hSosl-HA together with PKN. hsosl was immunoprecipitated with anti HA antibodies from each cell line, and the presence of PYK2 in the immunocomplexes was determined by immunobloting with anti-PYK2 antibodies. Expression levels of hsosl, PYK2 and PKN were determined by immunoblot analysis of total cell lysates, with anti-HA or anti PYK2 antibodies.

### Example 8

### PYK2 Induces Tyrosine Phosphorylation of Shc and its Association with Grb2

Activated EGF receptor is able to recruit Grb2 directly and indirectly. We have therefore investigated whether PYK2 can induce phosphorylation of Shc tyrosine phosphorviation of Shc and its association with Grb2. Shc proteins were immunoprecipitated with anti Shc antibodies from Shc, from Shc and PYK2, or from Shc and PKIN expressing cells. The samples were resolved by SDS-PAGE, transferred to nitrocellulose and immunoblotted with antiphosphotyrosine or anti-Grb2 antibodies. Dramatic tyrosine phosphorylation of Shc in cells that overexpress PYK2. Moreover, several phosphotyrosine containing proteins were found in Shc immunoprecipitates from PYK2 overexpressing cells. Similar results were observed in cells expressing endogenous Shc proteins that were transfected with PYK2 CDNA and subject to immunoprecipitation analysis with anti Shc antibodies. Immunoblot analysis with Grb2 antibodies of Shc immunoprecipitates indicated that Grb2 associates with tyrosine phosphorylated Shc in PYK2 overexpressing cells. We therefore conclude that tyrosine phosphorylated PYK2 can directly and indirectly recruit Grb2 via tyrosine phosphorylation of Shc revealing at least two alternative routes for PYK2 induced activation of the Ras signaling pathway.

Tyrosine phosphorylation of Shc in cells that coexpress PYK2 was standard. Cells that express Shc alone or coexpress Shc together with either PYK2, or PKN were lysed and subjected to immunoprecipitation with anti-Shc antibodies or pre-immune serum (P.I.). The immunocomplexes were washed, run on an SDS gel and immunoblotted with anti-phosphotyrosine antibodies. Shc proteins (46, 52 and 66 kDa) were identified.

PYK2 induces association of Shc with Grb2. Shc proteins were immunoprecipitated from each cell line using anti-Shc antibodies. As a control, the lysates of cells that coexpress PYK2 and Shc were subject to immunoprecipitation with pre-immune serum (P.I.). The presence of Grb2 in the immunocomplexes was determined by immunoblotting with anti-Grb2 antibodies.

The expression level of of PYK2, PKN and Shc in each cell line was determined by immunoblot analysis of total cell lysates with specific antibodies as indicated.

### Example 9

### Activation of MAP Kinase in PC12 Cells by Bradykinin, Carbachol and Other Stiumli

The experiments presented so far show that the same stimuli that induce activation of PYK2 induce tyrosine phosphorylationof Shc. We next examined the ability of these agents to induce the activation of kinases in PC12 cells. Quiescent PC12 cells were incubated with a variety of stimuli. Lysates from stimulated cells were subjected to immunoprecipitation with anti-MAP kinase antibodies followed by immunoblottinc, with phosphotyrosine antibodies. Myelin basic protein (MBP) was utilized as a substrate to determine MAP kinase activation. The addition of various ligands to PC12 cells induced a similar profile of both tyrosine phosphorylation and activation of MAP kinase in these cells.

Since activation of MAP kinase was observed in response to stimuli that induce PYK2 phosphorylation, we examined the possibility whether PYK2 overexpression can induce MAP kinase activation. Human embryonic kidney 293 cells were transiently transfected with increasing concentrations of mammalian expression vector that directs the synthesis of PYK2. The cells were grown for 24 hours in the presence of 0.2% serum, MAPK 1,2 proteins were immunoprecipitated, washed and subjected to MBP phosphorylation assay. The results presented in flaure 4b show that PYK2 overexpression induced MBP phosphorylation in a concentration dependent manner.

Quantitation of these results shows that MAP kinase activity was approximately three fold hicher in cells that expressed the hiahest level of PYK2 as compared to mock transfected cells.

293 cells were transiently transfected with mammalian expression vectors for Shc alone, Shc together with PYK2, or Shc toaether with PKN. PC12 cells were starved for 18 hours as described. The cells were stimulated for 5 min at 37 C with the indicated stimuli, lysed and subjected to immunoprecipitation with antiMAPK 1,2 antibodies (Santa Cruz Biotechnoloay, #c-14 and #c-16). The immunoprecipitates were washed twice with lysis buffer (Lev et al., Mol. Cell. Biol. 13, 2224-2234, 1993) and once with Tris-buffer containing 10 mM Tris-HCI pH 7.2, 100 mM NaCl, 1 mM Na-vanadate and 5 mM benzamidine. The immunocomplexes were resuspended in 40 μl of MAP kinase-buffer containing 30 mM Tris-HCI pH 8, 20 mM MgCl2, 2 mM MnCl₂, 15 μg, MBP, 10 μM ATP and 5 μCiτ-[³² P]ATP (Amersham). The samples were incubated for 30 min at 30° C. and the reactions were stopped by the addition of SDS-sample buffer. The samples were resolved on 15% SDS-PAGE and analysed by autoradiography. Human embryonic kidney 293 cells were trantsiently transfected with increasing concentration of pRK5-PYK2 DNA (0.5 μg). Twelve hours after transfection the cells were (grown in medium containing 0.2% serum for 24 hours. The cells were lysed, immunoprecipitated with MAPK 1,2 antibodies and subjected to MBP phosphorylation assay as describe above.

Quiescent PC12 cells were stimulated for 5 min at 37° C. with bradykinin (1 μM), carbachol (1 mM), KCl (75 mM), PMA (1.6 μM), NGF (100 mg/ml), or left unstimulated (−). The cells were lysed and MAPK 1,2 were immunoprecipitated with specific antibodies. The immunocomplexes were washed and either resolved by SDS-PAGE, transferred to nitrocellulose and immunoblotted with anti-phosphotyrosine antibodies, or subjected to a standard myelin basic protein (MBP) phosphorylation assay.

Activation of MAP kinase by overexpression of PYK2. Human embryonic kidney 293) cells were transiently transfected with increasing concentrations of a mammalian expression vector that directs the synthesis of PYK2. MAPK 1,2 proteins were immunoprecipitated from each cell line, the immunocomplexes were washed and subjected to MBP phosphorylation assay. Quantitation of MAP kinase activity for each cell line was determined by phosphorimager and ImagQuant software (Molecular Dynamics, Incorporated). MAPK activity in transfected cells is compared to activity detected in control mock transfected cells.

### Example 10

### Bradykinin Stimulation of PC12 Cells Induces Tyrosine Phosphorylation of PYK2

Ligand stimulation, immunoprecipitations and immunoblotting were performed. Chronic treatment with PMA was performed by incubation of the cells with 100 nM PMA for 12 hours at 37° C.

Time course of bradykinin induces tyrosine phosphorylation of PYK2. Quiescent PC12 cells were incubated at 37° C. with 1 μM bradykinin for indicated periods of time. PYK2 was immunoprecipitated from untreated (−) or treated cells, the immunocomplexes were washed, resolved by SDS-PAGE, transferred to nitrocellulose, and probed either with anti-phosphotyrosine or anti-PYK2 antibodies.

Quiescent PC12 cells were incubated with either 1 μM bradykinin (1 min at 37° C.) or with PMA (1.6 μM, 15 min at 37° C.) in the presence or absence of CaCl or EGTA (3 μM) as indicated. In some cases the cells were pretreated with 100 nM PMA for 12 h. PYK2 was immunoprecipitated from stimulated or unstimulated cells (−) and analysed by immunoblot analysis with either anti-phosphotyrosine or anti-PYK2 antibodies.

### Example 11

### Stimulation of Kv1.2 Potassium Channel Tyrosine Phosphorylation in Response to PYK2 Activation

We examined the possibility whether PYK2 can tyrosine phosphorylate the Kv1.2 channel and regulate its function. In order to test this possibility, we expressed in 293 cells the Kv1.2 protein, Kv1.2 together with PYK2, and as a control Kv1.2 with a kinase negative PYK2 mutant (PKN) or with the protein tyrosine kinase Fak. The cells were grown for 24 hours in medium containing 0.2% serum and then stimulated with PMA(1.6 μM), calcium ionophore (6 μM), or left unstimulated.

Immunoblotting analysis with phosphotyrosine antibodies following immunoprecipitation of PYK2, PKN and Fak by specific antibodies. PYK2 and Fak were phosphorylated on tyrosine even in unstimulated cells, and treatment with PMA induced tyrosine phosphorylation while treatment with calcium ionophore induced a weaker response. The level of expression of the kinase negative mutant of PYK2 (PKN) was similar to the expression of wild type PYK2 or FAK. Nevertheless, as expected, PKN was not found to be phosphorylated on tyrosine residues. We next analyzed the tyrosine phosphorylation of Kv1.2 channel in each cell line.

We have added to the cDNA expression construct of Kv1.2 an HA tag, and determined the level of Kv1.2 expression by immunoblot analysis with anti-HA antibodies. A similar amount of Kv1.2 protein was expressed in the transfected cell lines. The Kv1.2 protein was immunoprecipitated from unstimulated cells, as well as from, PMA or calcium ionophore stimulated cells. The immunoprecipitates were resolved by SDS-PAGE and immunobloted with anti-phosphotyrosine antibodies. Phosphorylation of Kv1.2 on tyrosine residues was observed only in cells co-expressing, PYK2. Moreover, tyrosine phosphor-ylation of Kv1.2 was enhanced by PMA or calcium ionophore treatments indicating that activation of PYK2 is required for PYK2 induced tyrosine phosphorylation of the potassium channel.

Embryonic human kidney 293 cells were transiently transfected with different combinations of mammalian expression vectors which direct the synthesis of Kv1.2-HA, PYK2, a kinase negative PYK2 (PKN) or the protein tyrosine kinase Fak. The cells were grown for 24 h in the presence of 0.2% serum and then either stimulated with PMA (1.6 μM, 10 min at 37° C.), the calcium ionophore A23187 (6 μM, 10 min at 37° C.) or left unstimulated (−).

Tyrosine phosphorylation of each protein was analysed following immunoprecipitation and immunoblotting with anti-phosphotyrosine antibodies. The expression of each protein was determined by immunoblot analysis of total cell lysates from each transfection with anti-PYK2, anti-HA or anti-Fak antibodies.

Tyrosine phosphorylation of Kv1.2 was analysed by immunoprecipitation of Kv1.2-HA protein from each cell line with anti-HA antibodies, followed by immunoblot analysis with anti-phosphotyrosine antibodies. 293 cells were transfected by the calcium phosphate technique as described (Wigler et al., Cell 16, 777-785, 2979). The influenza virus hemagglutinin peptide (YPYDVPDYAS) [SEQ. ID NO 22] tag was added to the C-terminal end of the Kv1.2 cDNA utilizing the following oligonucleotide primers in the PCR; ′5GCCAGCAGGCCATGTCACTGG-3′ [SEQ. ID NO 23] and ′5CGGAATTCTTACGATGCGTAGTCAGGGACATCGTATGGGTAGACATCAGTTAAC ATT TTG-′3 [SEQ. ID NO 24]. The PCR product was digested with BALI and EcoRI and used to substitute the corresponding fragment at the C-terminal end of the Kv1.2 cDNA. The Kv1.2-HA cDNA was subcloned into pRK5 downstream the CMV promotor.

A kinase negative mutant of PYK2 (PKN) was constructed by replacing Lys475 with an Ala residue by utilizing a site directed mutagenesis Kit (Clontech). The oligonucleotide sequence was designed to create a new NruI restriction site. The nucleotide sequence of the mutant was confirmed by DNA sequencing. The oligonucleotide sequence that used for mutagenesis is: ′5-CAATGTAGCTGTCGCGACCTGCAAGAAAGAC-3′[SEQ. ID NO 25] (NruI site—bold, Lys-AAC substituted to Ala-GCG underline). The full length cDNAs of PYK2, PKN and Fak were subcloned into the mammalian expression vectors pRK5 downstream to the CMV promotor.

### Example 12

### Suppression of Potassium Channel Action in Frog Oocytes by PYK2 Expression and PMA Treatment

In vitro capped RNA transcripts of Kv1.2, PYK2 and PKN were synthesized from linearized plasmids DNA templates utilizing the mMESSAGE mMACHINE kit (Ambion), following the supplier's protocols. The products of the transcription reaction (cRNAs) were diluted in RNAse-free water and stored at −70° C. Expression of the RNAs was done by injection of 50 nl of RNA into defolliculated stage V and VI oocytes from Xenopus laevis (Iverson et al., J. Neurosc. 10, 2903-2916, 1990). The injected oocytes were incubated for 2-3 days at 20° C. in L15 solution (1:2 dilution of Gibco's Leibovitz L15 medum in H20, with 50 U/ml nystatin, 0.1 mg/ml gentamycin, 30 mM HEPES buffer, pH 7.3-7.4, filtered through a 0.45 mm membrane). Electrophysiological Recording and analysis. Ionic currents were recorded with a two microelectrode voltage-clamp as described (Iverson et al., supra). The current were low-pass filtered KHz using an 8-pole Bessel filter and stored in a 80286 microcomputer using the pClamp acquisition system (Axon Instruments). The data was analyzed with the clamp fit pro-rams of the pCIamp system (Axon Instruments). All recording were performed at room temperature (20-230° C.). The recording chamber was continually perfused with recording solution. To avoid contamination of the oocyte by Ca+²-activated Cl⁻ currents low Cl⁻ recording solution was used (96 mM Na+, glutamate, 2 mM K⁺ glutamate, 0.5 mM CaCl₂, 5 mM MgCl₂, 5 mM HEPES buffer). The K+ currents were elicited in depolarizinc, steps from −100 to +40 mv in 10 mV increments every 15 seconds.

Kv1.2 currents from oocytes microinjected with either Kv1.2 mRNA, Kv1.2 and PYK2 mRNAs, or Kv1.2 and a kinase negative mutant of PYK2 mRNAs (PKN). Currents were elicited in response to depolarizing steps from −100 to +30 mV increments from a holding potential of −110 mV. Representative traces of Kv1.2 channels before and after bath application of 100 nM PMA at the annotated time (8 and 20 minutes) in the same cell.

Suppression of Kv1.2 currents in response to PMA is blocked by a kinase negative PYK2 mutant (PKN). Inhibition of Kv1.2 currents in an oocyte microinjected with Kv1.2 mRNA before and 25 minutes after treatment with PMA at 50 nM or 100 nM concentration. Recordings from an oocyte expression Kv1.2 and PYK2 or Kv1.2 and a kinase negative mutant of PYK2 under the same conditions as described above. The same protocol was utilized in both experiments.

We asked whether stimulation of PYK2 can suppress Kv1.2 currents. We explored the effect of PYK2 expression, on currents exhibited by Kv1.2 expression in Xenopus oocytes. Stage V oocytes were microinjected either with Kv1.2 transcripts or with Kv1.2 together with PYK2 or PKN mRNAs. Following two to three days of incubation at 20° C., macroscopic currents exhibited by the oocytes were recorded with a two microelectrode voltage clamp as described (Iverson et al., J. Neurosc. 10, 2903-2916, 1990). Outward rectifier currents were recorded upon membrane depolarization above −40 mV, indicating that a functional Kv1.2 channel is expressed in the oocytes. The expression of Kv1.2, PYK2 and PKN in the frog oocytes was confirmed by immunoblot analysis with anti-HA or anti-PYK2 antibodies.

We have examined the effect of PYK2 expression on Kv1.2 currents in oocytes in the absence or presence of PMA. We also examined the effect of the kinase negative mutant PKN on PMA induced suppression of Kv 1.2 currents mediated by the endogenous protein tyrosine kinase. Treatment of oocytes with PMA caused inhibition of Kv1.2 currents. As previously shown, the inhibition of the currents developed gradually after application of PMA reaching 80-90% inhibition after 20 min incubation (Huang et al., Cell 75, 1145-1156, 1993). Moreover, the rate of channel blockade was found to be dependent upon the concentration of PMA applied. Coexpression of PYK2 resulted in acceleration of Kv1.2 currents inhibition. Significant acceleration of current inhibition was observed at every concentration of PMA tested. For example, 8 min after the addition of 100 nM PMA, 25% inhibition of outward current was observed in oocytes expressing Kv1.2 alone as compared to 95% inhibition observed in oocytes coexpressing Kv1.2 and PYK2 proteins.

Current inhibition by PMA treatment in the absence or presence of PYK2 expression did not result in changes in both the kinetics or voltage dependence of the remaining currents Coexpression of Kv1.2 together with the kinase negative mutant of PYK2 (PKN) led to nearly complete inhibition of PMA induced potassium channel blockage. It is possible that the endogenous protein tyrosine kinase activated by PMA that is responsible for suppression of Kv1.2 currents in oocytes represents the xenopus homologue of PYK2 or a closely related protein tyrosine kinase that can be affected by a dominant interfering mutant of PYK2.

### Example 13

### PYK2 is Phosphorylated upon LPA and Bradykinin Stimulation of Cells

We have now demonstrated that lysophosphatidic acid (LPA) and bradykinin do in fact induce tyrosine phosphorylation of PYK2 as well as complex formation between PYK2 and activated Src. This observation provides novel strategies to screen for modulators of PYK2 signalling pathways. Moreover, tyrosine phosphorylation of PYK2 leads to binding of the SH2 domain of Src to the tyrosine at position 402 of PYK2 and activation of Src, thereby providing even further information useful in the rational design of such PYK2 signalling pathway modulators. Transient overexpression of a dominant interfering mutant of PYK2 or the protein tyrosine kinase Csk reduces LPA- or bradykinin-induced activation of MAP kinase. LPA- or bradykinin-induced MAP kinase activation was also inhibited by overexpression of dominant interfering mutants of Grb2 and Sos. Thus, without wishing to be bound to any particular theory of the invention, we propose that PYK2 acts in concert with Src to link Gi- and Gq-coupled receptors with Grb2 and Sos to activate the MAP kinase signalling pathway in PC12 cells.

PC12 cells were stimulated with lysophosphatidic acid (LPA) and lysates of stimulated or unstimulated cells were subjected to immunoprecipitation with antibodies against PYK2 followed by immunoblotting with antibodies against phosphotyrosine. The experiment shows rapid tyrosine phosphorylation of PYK2 in response to LPA or bradykinin stimulation. Pretreatment of PC12 cells with pertussis toxin significantly inhibited tyrosine phosphorylation of PYK2 in response to LPA stimulation. However, bradykinin or phorbol 12-myristate 13-acetate (PMA) induced activation of PYK2 were not affected by pretreatment with pertussis-toxin. Removal of extracellular calcium by the addition of EGTA to the medium did not affect LPA-induced PYK2 phosphorylation. These results indicate that LPA-induced PYK2 activation is dependent, at least in part, on a pertussis-toxin sensitive Gi-dependent pathway in PC12 cells.

PC12 cells were stimulated with LPA (2.5 mM) or bradykinin (1 mM) for 3 minutes at 37° C. and lysed. PYK2 was immunoprecipitated with antibodies against PYK2 and immunoblotted with antibodies against phosphotyrosine (anti-pTyr) or against PYK2.

PC12 cells were left untreated (−) or incubated with pertussis toxin (PTx) 100 ng/ml for 20 hours and then stimulated with LPA (2.5 mM), bradykinin (1 mM) for 3 min at 37° C. or with PMA (1 mM) for 10 min at 37° C. In addition, cells were stimulated with LPA in medium containing 3 mM EGTA. We have analyzed phosphorylation of immunoprecipitated PYK2 by immunoblotting with anti-pTyr or anti-PYK2 antibodies. LPA, PMA, PMA, and bradykinin yielded 4, 12, and 9 fold increases in the phosphorylation state of PYK2 relative to unstimulated cells.

PC12 cells were transiently transfected with pRK5, PYK2 kinase negative mutant (PKM) and Csk. Expression of Csk was determined by blotting total cell lysates with anti-Csk antibodies.

PC12 cells were mock transfected (pRK5), or transfected with truncated EGF receptor, PKM, Csk, or with both PKM and Csk, stimulated with LPA or bradykinin for 3 min, lysed and MAP kinase activity was determined. The transfection efficiency was determined using a b-Galactosidase transgene. Similar results were obtained in three independent experiments performed in duplicate.

MAP kinase activity in PC12 cells over-expressing dominant interfering mutants of Grb2 and Sos was also assessed. Cells were stimulated with LPA or bradykinin and MAP kinase activity was determined. The experiments were repeated three times.

### Example 14

### PYK2 and Src Associate upon LPA and Bradykinin Stimulation of Cells

We have demonstrated that activation of PYK2 by elevation of intracellular calcium concentration can lead to activation of the MAP kinase signaling pathway in PC12 cells. Src family protein tyrosine kinases are activated in response to stimulation of a variety of G protein-coupled receptors and have been shown to be necessary for linking Gi- and Gq-coupled receptors with MAP kinase activation. Sadoshima & Izumo, EMBO J. 15:775-787 (1996); Wan et al., Nature 380:541-544 (1996). The EGF-receptor and erbB2 were also implicated in MAP kinase activation induced by LPA and other agonists of G-protein coupled receptors. Daub et al., Nature 379:557-564 (1996). We have tested whether LPA and bradykinin can activate Src and the EGF receptor in PC12 cells. Stimulation of PC12 cells by LPA or bradykinin leads to approximately three to four fold increase in Src kinase activity, while we were unable to detect LPA-induced activation of EGF receptor in PC12 cells.

We examined the possibility of association between the two protein tyrosine kinases PYK2 and Src in response to LPA or bradykinin stimulation. Lysates from stimulated or unstimulated cells were subjected to immunoprecipitation with antibodies against Src followed by immunoblotting with antibodies against PYK2, against Src or anti-pY416src antibodies that specifically recognize activated Src. Liu et al., Oncogene 8:1119-1126 (1993). This experiment demonstrated that PYK2 forms a complex with activated Src in response to LPA or bradykinin stimulation. Furthermore, the SH2 domain of Src bound to activated PYK2 suggesting that Src binding to PYK2 is mediated by means of its SH2 domain perhaps leading to its activation.

To further analyze the interaction between Src and PYK2, we performed an in vitro binding experiment using a GST fusion protein containing the SH2 domain of Src with wild-type PYK2, a mutant form of PYK2 in which the tyrosine at position 402 was replaced by a phenylalanine residue (PYK2-Y402F) or a kinase negative mutant of PYK2. Lev et al., 1995, Nature 376:737-745. A GST fusion protein containing the SH2 domain of Src bound to tyrosine phosphorylated PYK2 but not to the PYK2-Y402F mutant or PKM. The tyrosine at position 402 represents the major autophosphorylation site of PYK2 and is found within the sequence YAEI, a consensus binding site for the SH2 domain of Src kinases Songyang et al., Cell 72:76-778 (1993).

PC12 cells were left untreated (−) or stimulated with LPA (2.5 mM) or bradykinin (1 mM) for 3 min at 37° C. Src was immunoprecipitated and immunoblotted with antibodies against Src, anti-pY416src antibodies or antibodies against PYK2.

The same lysates were mixed with a GST fusion protein containing the SH2 domain of Src and the bound proteins were analyzed by immunoblotting with antibodies against PYK2.

293T cells were transiently transfected with pRK5, PYK2, PYK2-Y402F and PKM. Total cell lysates were analyzed by immunoblotting with anti-pTyr or anti-PYK2 antibodies. The same lysates were subjected to immunoprecipitation with antibodies against src and immunoblotting with anti-pY416src or antibodies against Src. Src kinase activity was quantitated as an increase in pY416src phosphorylation and presented as mean +/− S.D. from four independent experiments.

The effects of coexpression of PYK2 and Csk on PYK2 tyrosine phosphorylation and MAP kinase activation were also assessed. pRK5, PYK2, PYK2-Y402F or PYK2 plus increasing concentrations of Csk were transiently transfected in 293T cells. Total cell lysates were analyzed by immunoblotting with antibodies against phosphotyrosine, PYK2 and Csk. A weak tyrosine phosphorylation of PYK2 Y402F was observed upon longer exposure. The same lysates were used to determine MAP kinase activation. This experiment was repeated three times. It was thus determined that PKY2 and Src are involved in MAP kinaseactivation.

### Example 15

### PYK2 and Src Activate MAPK

We further examined the status of Src phosphorylation and MAP kinase activation upon transfection of PYK2 or PYK2-Y402F in human embryonic kidney 293T cells. Overexpression of PYK2 leads to approximately four fold stimulation of endogenous Src activity in these cells. However, overexpression of PYK2-Y402F or a kinase negative mutant of PYK2 (PKM) did not affect Src activity in the same assay. These experiments demonstrated that autophosphorylation of PYK2 on Tyr402 leads to the binding of the SH2 domain of Src and subsequent Src activation.

We next overexpressed PYK2, PYK2-Y402F or PKM with increasing amounts of Csk, a protein tyrosine kinase that negatively regulates Src (Nada et al., Nature 351:69-72 (1991)), and tested whether PYK2-induced Src activation contributes to PYK2 tyrosine phosphorylation and PYK2-induced MAP kinase activation in 293T cells. The tyrosine phosphorylation of PYK2 and PYK2-induced MAP kinase activition, normally observed from cells overexpressing PYK2, were significantly reduced in the presence of increasing amounts of Csk. Lev et al., Nature 376: 737-745 (1995). In addition, MAP kinase activation was greatly reduced in cells overexpressing PYK2-Y402F as compared to MAP kinase activation induced by expression of wild-type PYK2. PYK2-Y402F is poorly tyrosine phosphorylated upon overexpression. Moreover, overexpression of PYK2-Y402F did not activate Src in these experiments. These experiments demonstrate that PYK2 tyrosine phosphorylation and PYK2-induced MAP kinase activation are dependent, at least in part, on Src kinase activity stimulated by binding to autophosphorylated tyr402 on PYK2.

We next examined the role of PYK2 in LPA- or bradykinin-induced MAP kinase activation by utilizing the dominant interfering kinase negative mutant of PYK2. Lev et al., Nature 376:737-745 (1995); Tokiwa et al., Science 273:792-794 (1996). We were unable to generate stable PC12 cell lines that overexpress PKM and therefore used a transient overexpression strategy. Overexpression of PKM in PC12 cells strongly inhibited MAP kinase activation by LPA or bradykinin. The role of Src in LPA- or bradykinin-induced MAP kinase activation was further tested by transient overexpression of Csk. When Csk was overexpressed in PC12 cells a strong reduction in LPA- or bradykinin-induced MAP kinase was observed. In cells that were co-transfected with both PKM and Csk, LPA- or bradykinin-induced MAP kinase activation was profoundly inhibited. However, overexpression of PKM or Csk did not affect EGF- or nerve growth factor-induced MAP kinase activation in PC12.

Taken together, these experiments reveal a specific role for PYK2 and Src in linking G-protein coupled receptor, but not growth factor receptors, with MAP kinase activation.

Although certain embodiments and examples have been used to describe the present invention, it will be apparent to those skilled in the art that changes to the embodiments and examples shown may be made without departing from the scope or spirit of the invention.

Those references not previously incorporated herein by reference, including both patent and non-patent references, are expressly incorporated herein by reference for all purposes.

Other embodiments are within the following claims.

What is claimed is:

1. Isolated, purified, or enriched nucleic acid encoding a nucleic acid encoding PYK2 polypeptide.

2. A nucleic acid probe for the detection of nucleic acid encoding a PYK2 polypeptide in a sample.

3. Recombinant nucleic acid encoding a PYK2 polypeptide and a vector or a promoter effective to initiate transcription in a host cell.

4. Recombinant nucleic acid comprising a transcriptional region functional in a cell, a sequence complimentary to an RNA sequence encoding a PYK2 polypeptide and a transcriptional termination region functional in a cell.

5. An isolated, purified, recombinant, or enriched PYK2 polypeptide having a phosphorylation activity.

6. A purified antibody having specific binding affinity to a PYK2 polypeptide.

7. hybridoma which produces an antibody having specific binding affinity to a PYK2 polypeptide.

8. A method of detecting a compound capable of binding to a PYK2 polypeptide comprising the steps of incubating said compound with said PYK2 polypeptide and detecting the presence of said compound bound to said PYK2 polypeptide.

9. The method of claim 8, wherein said compound inhibits a phosphorylation activity of said PYK2 polypeptide and is selected from the group consisting of tyrphostins, quinazolines, quinoxolines, oxindolinones, and quinolines.

10. A compound capable of inhibiting the phosphorylation activity of a P 2 polypeptide identified by the method of claim 9.

11. A method of screening potential agents useful for treatment of a disease or condition characterized by an abnormality in a signal transduction pathway, wherein said signal transduction pathway includes an interaction between a PYK2 polypeptide and a natural binding partner, comprising the step of assaying said potential agents for those able to promote or disrupt said interaction as an indication of a useful said agent.

12. A method for diagnosis of a disease or condition characterized by an abnormality in a signal transduction pathway, wherein said signal transduction pathway includes an interaction between a PYK2 polypeptide and a natural binding partner, comprising the step of detecting the level of said interaction as an indication of said disease or condition.

13. A method for treatment of an organism having a disease or condition characterized by an abnormality in a signal transduction pathway, wherein said signal transduction pathway includes an interaction between a PYK2 polypeptide and a natural binding partner comprising the step of promoting or disrupting said interaction.

14. The method of claim 13, wherein said disease or condition is selected from the group consisting of epilepsy, schizophrenia, extreme hyperactivity in children, chronic pain and acute pain.

15. The method of claim 13, wherein said disease or condition is selected from the group consisting of stroke, alzheimer's disease, parkinson's disease, neurodegenerative diseases, and migraine.

16. The method of any one of claims 11-15, wherein said natural binding partner is Src, and wherein said pathway involves a G protein-coupled receptor, but does not involve a growth factor receptor.

17. An isolated, purified, or enriched nuclic acid sequence that:
(a) encodes a polypeptide having the full length amino acid sequence set forth in SEQ ID NO: 2;
(b) the complement of the nucleotide sequence of (a);
(c) hybridizes under highly stringent hybridization conditions to the nucleotide sequence of (a) and encodes a naturally occurring PYK2 protein;
(d) enocdes a PYK2 protein having the full length amino acid sequence set forth on SEQ ID NO: 2 except that it lacks a segment of amino acid residues selected from the group consisting of: 1-417, 418-679, 713-733, 843-860, and 861-1009;
(e) the complement of the nucleotide sequence of (d);
(f) encodes a polypeptide having an amino acid sequence selected from the group cositing of amino acid residues; 1-417, 418-679, 713-733, 843-860, and 861-1009;
(g) the complement of the nucleotide sequence of (f);
(h) encodes a polypeptide having the full length amino acid sequence set forth in SEQ ID NO: 2 except that it lacks at least one, but not more than two, of the domains selected from the group consisting of the N-terminal domain, the kinase domain, the first protein rich domain, the second protein rich domain, and the FAT targeting domain; or
(i) the complement of the nucleotide sequence of (h).

18. A recombinant vector containing the nucleotide sequence of claim 17.

19. A genetically engineered host cell containing the nucleotide sequence of claim 17.
